(19) United States
(12) Patent Application Publication
(10) Pub. No.: US 2016/0271248 A1
(43) Pub. Date: Sep. 22, 2016

(54) CXCR4 UP- AND DOWN-REGULATION FOR TREATMENT OF DISEASES OR DISORDERS

(71) Applicant: DREXEL UNIVERSITY, Philadelphia, PA (US)

(72) Inventor: Olimpia MEUCCI, Penn Valley, PA (US)

(21) Appl. No.: 14/917,614

(22) PCT Filed: Oct. 3, 2014
(86) PCT No.: PCT/US14/59107

§ 371 Date: Mar. 9, 2016

Related U.S. Application Data
(60) Provisional application No. 61/886,371, filed on Oct. 3, 2013.

(51) Int. Cl. A61K 39/395; C07K 16/24; A61K 45/06; A61K 31/485

(52) U.S. Cl. CPC A61K 39/3955; A61K 31/485; C07K 16/245; A61K 45/06; C07K 2317/76

(57) ABSTRACT

The present invention includes a method of treating or preventing HAND in a subject in need thereof, wherein the method comprises administering to the subject a composition that down-regulates ferritin heavy chain (FHC). The present invention further includes a method of treating or preventing HAND in a subject in need thereof, wherein the method comprises administering to the subject a composition that decreases the concentration, expression level and/or activity of IL-1β. The present invention further includes a method of treating or preventing in a subject in need thereof a disease or condition associated with CXCR4 up-regulation, such as but not limited to cancer, metastasis, liver fibrosis (including HIV-associated liver fibrosis), or HIV infection, wherein the method comprises administering to the subject a composition that up-regulates FHC.

### CROSS-REFERENCE TO RELATED APPLICATIONS

The present application claims priority under 35 U.S.C. §119(e) to U.S. Provisional Patent Application No. 61/886,371, filed Oct. 3, 2013, which application is incorporated herein by reference in its entirety.

### STATEMENT REGARDING FEDERALLY SPONSORED RESEARCH OR DEVELOPMENT

This invention was made with government support under grant numbers DA15014, DA32444 and MH097623 awarded by National Institutes of Health. The government has certain rights in the invention.

### BACKGROUND OF THE INVENTION

The chemokine CXCL12 and its cognate receptor CXCR4 perform multiple functions essential for the development and function of the central nervous system, including guiding the migration and differentiation of neuronal precursor cells, regulating cell cycle proteins, and modulating NMDA receptor signaling. These homeostatic functions take on added significance within the context of neuroinflammatory disease, as the CXCL12/CXCR4 signaling axis aids recovery by promoting survival of mature neurons, recruitment of neural and glial progenitor cells, and neuronal-glial communication.

Among the numerous etiologies of neuroinflammatory disease, CXCR4 dysfunction may be particularly relevant in the context of HIV associated neurocognitive disorders (HAND). Due to its role as an HIV coreceptor and its expression in both neuronal and non-neuronal cells, CXCR4 has been directly implicated in HIV infection and neuropathogenesis. The neuropathology of HAND is complex, although consistent findings associated with severe forms include an elevation of inflammatory cytokines, high levels of excitotoxins, neuronal loss, and decreased synaptic density. HAND is both accelerated and complicated by the frequent comorbidity of illicit drug abuse, primarily intravenous opiate abuse; when compared to non-drug-abusing HIV patients, drug abuse increases the frequency of HIV encephalitis, enhances microglia activation, promotes giant cell formation, and increases blood brain barrier disruption.

Opiates, including morphine (the major metabolite of heroin, which easily reaches the brain), alter CXCR4 function in immune and neural cells. Further, morphine and selective μ-opioid agonists, such as DAMGO (also known as [D-Ala², N-MePhe⁴, Gly-ol]-enkephalin), specifically impairs CXCL12/CXCR4 signaling in neurons, both in vitro and in vivo, by increasing protein levels of ferritin heavy chain (FHC), a recently described CXCR4 regulator. Morphine increases FHC levels and its association with CXCR4, thereby inhibiting CXCR4 activation and downstream pro-survival signaling pathways. This novel CXCR4-regulatory function of FHC occurs seemingly independent from the primary role of the protein as a critical regulator of intracellular iron levels. In this context FHC functions by binding, oxidizing, and sequestering free reactive iron, which is stably stored within the ferritin complex formed by FHC and its partner subunit ferritin light chain (FLC). Ferritin's ferroxidase activity is a critical property of FHC, responsible for oxidizing free elemental Fe²⁺ to the less reactive Fe³⁺, and is essential in controlling the production of toxic free radicals. Within the CNS, FHC and FLC proteins can be found in all cell types but their relative ratio varies between neuronal and glial cells. FHC is predominant in oligodendrocytes, microglia and neurons. FHC production is regulated in response to changing iron levels and inflammatory processes.

There is a need in the art to identify a method of treating HAND. Further, there is a need in the art to identify a method of treating conditions associated with CXCR4 up-regulation, including cancer, metastasis, liver fibrosis (including HIV-associated liver fibrosis), HIV infection or pain. The present invention fulfills these needs.

### BRIEF SUMMARY OF THE INVENTION

The invention provides a method of treating or preventing HAND in a subject in need thereof. The invention further provides a method of treating or preventing in a subject in need thereof a disease or condition associated with CXCR4 up-regulation.

In certain embodiments, the method comprises administering to the subject a therapeutically effective amount of an agent that down-regulates FHC in the subject, whereby HAND is treated or prevented in the subject.

In certain embodiments, the method comprises administering to the subject a therapeutically effective amount of an agent that decreases that concentration, expression level and/or activity of IL-1β in the subject, whereby HAND is treated or prevented in the subject.

In certain embodiments, the method comprises administering to the subject a therapeutically effective amount of an opioid agonist, whereby the disease or condition is treated or prevented.

In certain embodiments, the agent is selected from the group consisting of an antibody, siRNA, ribozyme, antisense, aptamer, peptidomimetic, iron chelating agent, and any combinations thereof. In other embodiments, the antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

In certain embodiments, the agent is at least one selected from the group consisting of interleukin-1 receptor antagonist (IL-1ra), anakinra and rilonacept.

In certain embodiments, the disease or condition comprises cancer, metastasis, liver fibrosis, HIV infection or pain. In other embodiments, liver fibrosis includes HIV-associated liver fibrosis. In yet other embodiments, the opioid agonist is at least one selected from the group consisting of adrenorphin, amidorphin, casomorphin, DADLE, DAMGO, dermorphin, endomorphin, morphiceptin, octreotide, opiorphin, TRIMU 5, codeine, morphine, thebaine, oripavine, esters of morphine, ethers of morphine, semi-synthetic alkaloid derivatives, anilidopiperidines, phenylpiperidines, diphenylpropylamine derivatives, benzomorphan derivatives, oripavine derivatives, morphinan derivatives, lefetamine, menthol, meptazinol, mitragynine, tilidine, tramadol and tapentadol.

In certain embodiments, the subject is a mammal. In other embodiments, the mammal is human.

In certain embodiments, the agent is administered by an inhalational, oral, rectal, vaginal, parenteral, topical, transdermal, pulmonary, intranasal, buccal, ophthalmic, intrathecal, intracranial, or intravenous route of administration. In other embodiments, the opioid antagonist is administered by an inhalational, oral, rectal, vaginal, parenteral, topical, transdermal, pulmonary, intranasal, buccal, ophthalmic, intrathecal, intracranial or intravenous route of administration.

In certain embodiments, the subject is further administered at least one anti-HIV drug. In other embodiments, the disease or condition comprises HIV infection and the subject is further administered at least one anti-HIV drug. In yet other embodiments, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1E, illustrates the finding that HIV infection and opiate drug use (DU) associate with increased levels of FHC and decreased pCXCR4 within neurons of the prefrontal cortex in human HIV+ and DU patients. The optical density (OD) of FHC (FIG. 1A), phosphorylated (active) CXCR4 (FIG. 1B), and total CXCR4 (FIG. 1C) within MAP2+ neurons was quantified among four patient groups: Control (HIV−/DU−), DU (HIV−/DU+), HIV (HIV+/DU−) and HIV/DU (HIV+/DU+). Approximately 100 neurons were analyzed for each patient, with patient numbers as follows: Control (7-14), DU (5-7, HIV (5-16), HIV/DU (3-8). Left panels indicate mean OD for each neuron, and right panels show average neuronal OD for each patient group; *P<0.05, **P<0.01, ***P<0.001. As illustrated in FIG. 1D, plotting each patient's average FHC OD to that of pCXCR4 revealed a significant inverse correlation, indicating a negative relationship between FHC expression and CXCR4 activation; n=16, Pearson Correlation=−0.724, P=0.002. As illustrated in FIG. 1E, no significant relationship was found between average neuronal FHC OD and age at death for any patient group.

FIG. 2, comprising FIGS. 2A-2E, illustrates the finding that SIV infection and morphine treatment associate with increased levels of FHC and decreased pCXCR4 within PFC neurons in rhesus macaques. The optical density (OD) of FHC (FIG. 2A), phosphorylated (active) CXCR4 (FIG. 2B), and total CXCR4 (FIG. 2C) within MAP2+ neurons was quantified among four treatment groups: Control (SIV−/Morphine−), Morphine (SIV−/Morphine+), SIV (SIV+/Morphine−) and SIV/Morphine (SIV+/Morphine+); 100 neurons were analyzed for each animal, with animal numbers as follows: Control—7, Morphine—3, SIV—4, SIV/Morphine—2. Left panels indicate mean OD for each neuron, and right panels show average neuronal OD for each treatment group; *P<0.05, **P<0.01 (Statistical analysis includes Control, Morphine and SIV groups only; SIV/Morphine animals were not included as n=2). As illustrated in FIG. 2D, plotting each animal's average FHC OD to that of pCXCR4 reveals a significant inverse correlation, indicating a negative relationship between FHC expression and CXCR4 activation; n=15, Pearson Correlation=−0.547, P=0.035. As illustrated in FIG. 2E, among the 4 SIV+/Morphine− macaques, FHC and pCXCR4 levels were apparently stratified, such that 2 animals expressed higher levels of FHC and lower levels of pCXCR4 (red rectangle), and 2 animals expressed lower levels of FHC and higher levels of pCXCR4 (blue rectangle). Clinical data identified the animals with greater FHC as “rapid progressors,” retaining an elevated viral load after infection, suggesting a relationship between FHC induction and disease severity.

FIG. 3, comprising FIGS. 3A-3B, illustrates the finding that wild-type (WT) and mutant 222 (Mut) FHC similarly inhibit CXCR4 signaling in HOS cells. (FIG. 3A) WT and Mut (non-iron-binding) FHC or empty vector (EV) were transfected into HOS cells (left panel) and the specific inability of mutant FHC to bind iron was confirmed by calcein assay (right panel), a measure of free intracellular iron levels. Calcein fluorescence was increased by treatment with the iron chelator desferrioxamine (DFO), or by over-expressing WT FHC, but not by expressing Mut FHC. As illustrated in FIG. 3B, effects of these proteins on CXCL12 signaling were compared by treating cells with 20 nM CXCL12 for various times, as indicated. Both WT and Mut FHC expressing cells demonstrate a similar reduction in CXCL12-induced Akt and CXCR4 activation; n=4, *P<0.05, #P<0.01, ̂P<0.001 as compared to each respective empty vector control.

FIG. 4, comprising FIGS. 4A-4F, illustrate the finding that CXCL12 regulates dendritic spine density in vitro and in vivo. As illustrated in FIG. 4A, rat cortical neurons cultured for 21 days in presence of a glial feeder layer were treated with CXCL12 (20 nM, 3 hours), resulting in increased dendritic spine density; n=12, P<0.001. As illustrated in FIG. 4B, rat cortical neurons cultured for 21 days as pure neuronal population were treated with 20 nM CXCL12, resulting in time-dependent increase in dendritic spines; n=7-29, green data P<0.05. As illustrated in FIG. 4C, this type of treatment also increased nuclear levels of HDAC4 in pure neuronal cultures, as visualized by immunocytochemistry (left panel, 30 minutes CXCL12 treatment), and quantified as differences in average HDAC4 nuclear pixel intensity (right panel); n=12-34, green data P<0.05. Similar results were obtained by western blot using nuclear extracts of control and CXCL12-treated (20 nM; 30 min) neurons, as shown in the bottom panel. As illustrated in FIG. 4D, a pure neuronal population was transfected with siRNA after 8 days in culture, resulting in specific decrease in HDAC4, but not HDAC1 36 hours after transfection (upper panel). HDAC4 deficiency completely blocked the effects of CXCL12 on dendritic spines (lower panel); n=8-17, green data P<0.05. As illustrated in FIG. 4E, the CXCR4 antagonist AMD3100 (also known as 1,1′-[1,4-phenylene bis(methylene)]bis[1,4,8,11-tetraazacyclotetradecane]), delivered by ICV injection, decreases dendritic spine density in vivo. 1 μg of AMD3100 was injected into the left lateral ventricle of 3-week-old rats, and analysis of layer II/III pyramidal neurons of animals sacrificed 6 hours post-injection revealed a reduction in total dendritic spine density; n=15, P<0.01. As illustrated in FIG. 4F, this effect of AMD3100 was similarly confirmed in a prolonged treatment model; 3-week-old rats were implanted with osmotic pumps delivering constant (0.75 fig/hour) levels of AMD3100 for 4 days, which resulted in decreased total dendritic spine density; n=15, P<0.01.

FIG. 5, comprising FIGS. 5A-5D, illustrates the finding that regulation of the CXCR4 axis alters the frequency and amplitude of EPSCs of layer 2/3 pyramidal neurons. As illustrated in FIG. 5A, summary graphs show that ICV injection of AMD3100 has no effect on sEPSC frequency or amplitude (p>0.05 for both). As illustrated in FIG. 5B, AMD3100 exposure significantly decreases mEPSC frequency (p<0.04) but not amplitude (p>0.05). Upper panel, sample traces of mEPSCs recorded at −70 mV in the presence of TTX (1 μM) and picrotoxin (100 μM) in layer 2/3 pyramidal neurons from vehicle and AMD3100 exposed animals. Lower panel, summary graphs showing the mEPSC frequency and amplitude. As illustrated in FIG. 5C, in contrast, summary graphs show CXCL12 (20 nM, 3 hrs) incubation of prefrontal cortical slices induced no change in sEPSC frequency (p>0.05), but significantly increased sEPSC amplitude (p<0.03). As illustrated in FIG. 5D, CXCL12 exposure had no significant effect on mEPSC frequency or amplitude (p>0.05 for both). Upper panel, sample traces of mEPSCs recorded at −70 mV in the presence of TTX (1 μM) and picrotoxin (100 μM) in layer 2/3 pyramidal neurons from vehicle and CXCL12 exposed slices. Lower panel, summary graphs showing the mEPSC frequency and amplitude.

FIG. 6, comprising FIGS. 6A-6F, illustrates the finding that morphine regulates dendritic spine density through effects on neuronal FHC. Rat cortical neurons cultured for 21 days were treated with morphine (1 μm, 24 hours), resulting in both (FIG. 6A) increased FHC levels (n=4, P<0.001) and (FIG. 6B) decreased dendritic spine density (n=12, P<0.05). Similarly, 3-week-old rats treated with sustained-release morphine pellets for 4 days exhibited (FIG. 6C) increased brain FHC levels (n=4, P<0.001) and (FIG. 6D) decreased total dendritic spine density (n=12, P<0.05). As illustrated in FIG. 6E, FHC over-expression in the absence of morphine also causes a decrease in dendritic spine density, as measured in cultured rat cortical neurons transfected with a FHC-expressing plasmid or empty vector (n=16, P<0.001). As illustrated in FIG. 6F, the ability of morphine to regulate dendritic spine density is blocked in FHC-deficient neurons, as was shown using FHC-specific or scrambled (control) shRNA.

FIG. 7 is a set of photographs, illustrating the semi-quantitative measure of FHC protein within MAP2+ neurons using multispectral imaging. Individual cortical neurons are digitally identified and isolated from frontal cortex tissue using the optical density (OD) of the MAP2:NovaRed complex. The neuronal map was superimposed over the corresponding OD of the FHC:VectorBlue spectrum. Optical densities of both MAP2 and FHC are combined in a pseudocolored composite image. Both the macaque morphine treatment group and the human drug use group show greater FHC protein expression within cortical neurons than the respective control groups. (RGB: red green blue image, MAP2: optical density image of MAP2:NovaRed conjugated chromogen, FHC: optical density of FHC:Vector:Blue conjugated chromogen. Composite: pseudocolored image of MAP2 (red) and FHC (blue) optical densities. All Images at 40× Magnification.

FIG. 8 is a set of graphs illustrating the finding that CXCR4 antagonist AMD3100 and pertussis toxin inhibit the effect of CXCL12 on dendritic spine density. Rat cortical neurons cultured for 21 days were pre-treated with either AMD3100 (100 ng/ml; added 20 min before addition of CXCL12) or Pertussis Toxin (PTx, 100 ng/ml; 18 hrs before addition of CXCL12) and then exposed to CXCL12 (20 nM, 3 hours). Data reported in light green are significantly different than controls (P<0.05; One way ANOVA followed by Dunnett test).

FIG. 9, comprising FIGS. 9A-9C, is a series of photographs illustrating technical approaches for in vivo experiments. As illustrated in FIG. 9A, methods for in vivo dendritic spine analysis. Rat brains were stained using a Golgi stain kit, as described in the methods. This system selectively impregnates a subset of neurons, in a likely random manner. Individual neurons from layer II/III of the prefrontal cortex were reconstructed in their entirety, first by tracing the dendrites, then by indicating individual dendritic spines. This process creates a 3-dimensional map of each neuron, including fine morphological details. As illustrated in FIG. 9B, stereotaxic brain injections target the ventricular system. In order to confirm the correct targeting of the ICV injections, 5 μL of 2% Evans blue dye were injected into the left lateral ventricle and verified distribution of the dye. FIG. 9C illustrates neurons.

FIG. 10, comprising FIGS. 10A-10B, illustrates the results of the experiment wherein rat cortical neurons were exposed to either TNF-α or IL-1β in the absence of glia. FIG. 10A is a bar graph that illustrates the fold increase in FHC protein levels compared to vehicle-treated controls. FIG. 10B is a bar graph that illustrates FHC transcript levels increased following agent treatment.

FIG. 11, comprising FIGS. 11A-11C, illustrates the finding that the HIV-1 envelope protein, gp120, modulates neuronal FHC in a temporal manner. FIG. 11A: gp120_{IIIB} was able to only transiently alter levels of FHC in pure neuronal cultures. FIG. 11B: gp120_{BaL}, a CCR5-binding viral protein, failed to upregulate neuronal FHC in the absence of glia cells. FIG. 11C: Upon exposure to gp120_{IIIB} in the presence of glia, neuronal expression of FHC protein was significantly higher compared to vehicle-treated controls.

FIG. 12 is a set of bar graphs illustrating the finding that X4- and R5-tropic gp120 induced secretion of IL-1β and TNF-α in neuronal/glial co-cultures.

FIG. 13, comprising FIGS. 13A-13C, is a set of bar graphs and images illustrating that the presence of an IL-1β neutralizing antibody or IL-1 receptor antagonist abrogated gp120-mediated increases in FHC. Rat neurons cultured with a feeder layer of glia (8 DIV) were treated with either gp120_{IIIB} (200 pM) or gp120_{BaL} (200 pM) in the presence or absence of TNF-α antibody (40 μg/mL), IL-1β antibody (50 μg/mL) or IL-1ra (1 μg/mL). The presence of a TNF-α neutralizing antibody had no effect on the ability of gp120 to upregulate neuronal FHC. Neutralization or antagonism of IL-1β mitigated increases in neuronal FHC caused by gp120 in bilaminar cultures. Treatment with either the IL-1β antibody or IL-1ra alone had no significant effect on FHC protein levels (n=3; ***p<0.001 compared to vehicle; bars expressed as mean±SEM).

FIG. 14, comprising FIGS. 14A-14E, illustrates the finding that dendritic spine density is reduced in layer II/III pyramidal neurons of the PFC in two animal models of HAND and is associated with increased levels of FHC in HIV-Tg rats. Dendritic spine density was analyzed in control F344 (n=4), HIV-Tg (n=4), BSA-treated SD (n=6), and gp120-treated SD (n=5) rats using Golgi staining. Layer II/III pyramidal neurons of the prefrontal cortex (n=4 neurons per animal) were assessed using Neurolucida software. Both HIV-Tg and gp120-treated rats had significantly reduced dendritic spine density compared to their respective control animals (***p<0.001). Additionally, levels of FHC were increased in HIV-Tg rat brains compared to controls in the hemisphere contralateral to the one processed for dendritic spine analysis (*p<0.05; bars expressed as mean±SEM).

FIG. 15, comprising FIGS. 15A-15D, illustrates the finding that apical and basal dendritic spine density are reduced by similar magnitudes but only basal dendrite branching is decreased. Dendritic spine density was examined in apical and basal dendrites separately to assess whether one was preferentially downregulated. In both HIV-Tg and gp120-treated animals, basal and apical dendritic spine density were significantly decreased by similar extents, suggesting that there is no difference in susceptibility between the different dendrite types to incur spine injury. However, only branching of basal dendrites was reduced in both HIV-Tg and gp120-treated rats. A decrease in branching suggests that there is a decrease in neuronal complexity and the ability of layer II/III pyramidal neurons to receive excitatory inputs from other brain regions. (***p<0.001; *p<0.05; bars expressed as mean±SEM).

FIG. 16 is a bar graph illustrating the finding that SD rats treated with gp120 exhibit deficits relative to BSA treated controls in Reversals 2 and 3, as well as the extra-dimensional shift.

### DETAILED DESCRIPTION OF THE INVENTION

The present invention relates in one aspect to the unexpected discovery that opiates and HIV infection modulate the expression of the protein ferritin heavy chain (FHC) and the downstream activity of the chemokine receptor CXCR4.

The present disclosure supports a convergence of HIV and opiate exposure on increasing FHC, which may contribute to neuronal dysfunction through effects on CXCR4 signaling. Illicit opiate abuse contributes to the exacerbated neurological impairment seen in HIV+ individuals through FHC-dependent disruption of neuronal CXCL12/CXCR4 signaling. In certain embodiments, neuronal damage comprises a loss of dendritic spines, which is a highly consistent and likely causative event in the development of HIV-associated neurocognitive disorders (HAND). As described herein, opiates and HIV were shown to negatively regulate neuronal CXCL12/CXCR4 signaling within human and non-human primates, and decreased dendritic spine density was observed as a consequence of FHC induction. Together, these data provide the first evidence of regulation of dendritic spine by CXCL12/CXCR4 (with consequent functional changes in neuronal activity), and disclose a novel mechanism in the neuropathogenesis and neurocognitive impairment seen in drug-abusing HIV patients.

As demonstrated herein, viral or host components also contribute to FHC-mediated neuronal alterations, and subsequent neurocognitive impairment, observed in HIV-infected individuals. The present studies identify host components in response to HIV infection, specifically TNF-α and IL-1β, as major players in the increase of neuronal FHC. Additionally, by using a non-infectious animal model of HIV neuropathology, the HIV-1 transgenic rat, and animals treated with HIV-1 envelope protein gp120, the role of specific viral proteins in the induction of FHC and dendritic changes in vivo and in the resulting neurocognitive impairment was confirmed. The present studies quantify dendritic changes in the prefrontal cortex of these two HAND models and to show a direct (i.e., within the same group of animals) correlation of these structural changes with specific biochemical and functional alterations. Furthermore, the study demonstrates the pivotal role of IL-1β in the modulation of FHC by HIV-1 gp120.

In one aspect, the invention includes a method of treating or preventing HAND in a subject in need thereof, wherein the method comprises administering to the subject a therapeutically effective amount of a composition that down-regulates FHC.

In another aspect, the invention includes a method of treating or preventing HAND in a subject in need thereof, wherein the method comprises administering to the subject a therapeutically effective amount of a composition that decreases the concentration, expression level and/or activity of IL-1β.

In yet another aspect, the invention includes a method of treating or preventing in a subject in need thereof a disease or condition associated with CXCR4 up-regulation, wherein the method comprises administering to the subject a therapeutically effective amount of a FHC up-regulator. In certain embodiments, the FHC up-regulator comprises an opioid agonist. In other embodiments, the disease or condition includes cancer, metastasis, liver fibrosis (including HIV-associated liver fibrosis), HIV infection or pain.

### DEFINITIONS

As used herein, each of the following terms have the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics and chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, ±5%, ±1%, or ±0.1% from the specified value, as such variations are appropriate to perform the disclosed methods.

As used herein, the term “HAND” refers to any of HIV associated neurocognitive disorder. In certain embodiments, HAND includes HIV-associated dementia (HAD), asymptomatic neurocognitive impairment (ACI) and/or minor neurocognitive disorder (MND).

As used herein, the term “DAMGO” refers to [D-Ala², N-MePhe⁴, Gly-ol]-enkephalin or (2S)-2-[[2-[[(2R)-2-[[(2S)-2-amino-3-(4-hydroxyphenyl)propanoyl]amino]propanoyl]amino]acetyl]-methylamino]-N-(2-hydroxyethyl)-3-phenyl propanamide or a salt thereof.

As used herein, the term “FHC” refers to ferritin heavy chain.

As used herein, the term “FLC” refers to ferritin light chain.

As used herein, the term “down-regulation” or “downregulation” as relating to a protein, ligand and/or receptor refers to any process or mechanism that reduces, or slows or prevents the increase of, the concentration, expression level and/or activity of the protein, ligand and/or receptor. The term “down-regulation” or “downregulation” includes any process or mechanism described herein or known to those skilled in the art, including in vivo or ex vivo methods.

As used herein, the term “DU” refers to drug use.

As used herein, the term “OD” refers to optical density.

As used herein, the term “DFO” refers to desferrioxamine.

As used herein, the term “AMD3100” refers to 1,1′-[1,4-phenylenebis (methylene)]bis[1,4,8,11-tetraazacyclotetradecane] or a salt thereof.

As used herein, the term “PTx” refers to pertussis toxin.

As used herein, a “subject” refers to a human or non-human mammal. Non-human mammals include, for example, livestock and pets, such as ovine, bovine, porcine, canine, feline and murine mammals. In certain embodiments, the subject is human.

As used herein, the term “virus” is defined as a particle consisting of nucleic acid (RNA or DNA) enclosed in a protein coat, with or without an outer lipid envelope, which is capable of replicating within a whole cell.

The term “RNA” as used herein is defined as ribonucleic acid.

The term “recombinant DNA” as used herein is defined as DNA produced by joining pieces of DNA from different sources.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus. As used herein, a “peptidomimetic” is a compound containing non-peptidic structural elements that is capable of mimicking the biological action of a parent peptide. A peptidomimetic may or may not comprise peptide bonds.

The term “recombinant polypeptide” as used herein is defined as a polypeptide produced by using recombinant DNA methods.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein “endogenous” refers to any material from or produced inside an organism, cell, tissue or system. As used herein, the term “exogenous” refers to any material introduced from or produced outside an organism, cell, tissue or system.

“Isolated” means altered or removed from the natural state. For example, a nucleic acid or a peptide naturally present in a living animal is not “isolated,” but the same nucleic acid or peptide partially or completely separated from the co-existing materials of its natural state is “isolated.” An isolated nucleic acid or protein may exist in substantially purified form, or may exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues that are not present in the mature protein encoded by the mRNA molecule (e.g., amino acid residues in a protein export signal sequence).

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “antibody,” as used herein, refers to an immunoglobulin molecule that specifically binds with an antigen. Antibodies may be intact immunoglobulins derived from natural sources or from recombinant sources and may be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “immunoglobulin” or “Ig” is defined as a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitor-urinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “epitope” as used herein is defined as a small chemical molecule on an antigen that may elicit an immune response, inducing B and/or T cell responses. An antigen may have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

The term “expression” as used herein is defined as the transcription and/or translation of a particular nucleotide sequence driven by its promoter.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers may be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes may be labeled with, e.g., chromogenic, radioactive, or fluorescent moieties and used as detectable moieties.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

A “constitutive” promoter is a nucleotide sequence that, when operably linked with a polynucleotide that encodes or specifies a gene product, causes the gene product to be produced in a cell under most or all physiological conditions of the cell.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

The term “vaccine” as used herein is defined as a material used to provoke an immune response after administration of the material to a mammal.

As used herein, “vaccination” is intended for prophylactic or therapeutic vaccination.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “treatment” or “treating” is defined as the application or administration of a therapeutic agent, i.e., a composition useful within the invention (alone or in combination with another pharmaceutical agent), to a subject, or application or administration of a therapeutic agent to an isolated tissue or cell line from a subject (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder or the potential to develop the disease or disorder. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “modulate” is meant to refer to any change in biological state, i.e. increasing, decreasing, and the like. For example, the term “modulate” may be construed to refer to the ability to regulate positively or negatively the expression, stability or activity of FHC, including but not limited to transcription of a FHC mRNA, stability of a FHC mRNA, translation of a FHC mRNA, FHC stability, FHC post-translational modifications, FHC activity, or any combination thereof. Further, the term modulate may be used to refer to an increase, decrease, masking, altering, overriding or restoring of activity, including but not limited to, FHC activity.

As used herein, the terms “effective amount” or “therapeutically effective amount” or “pharmaceutically effective amount” of a compound are used interchangeably to refer to the amount of the compound which is sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced. An appropriate therapeutic amount in any individual case may be determined by one of ordinary skill in the art using routine experimentation.

“Parenteral” administration of a composition includes, e.g., subcutaneous (s.c.), intravenous (i.v.), intramuscular (i.m.), or intrasternal injection, or infusion techniques.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, intracranial and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

By the term “applicator,” as the term is used herein, is meant any device including, but not limited to, a hypodermic syringe, a pipette, and the like, for administering the compounds and compositions of the invention.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DISCLOSURE

The present disclosure provides the first systematic characterization of FHC expression in human neurons, and identifies a novel mechanism contributing to neuronal dysfunction caused by opiate use and HIV infection. Increased levels of FHC were observed within neurons of the prefrontal cortex associated with both opiate drug use and HIV/SIV infection in human and rhesus macaque tissue. FHC levels across treatment groups were shown to negatively correlate with pCXCR4 levels, suggesting that pathological induction of FHC, whether caused by opiate exposure or viral infection, similarly inhibits CXCR4 signaling. A novel neuroprotective activity of CXCR4 in its ability to increase dendritic spine numbers was characterized, and FHC was implicated in morphine-induced spine loss, an important pathological component of HAND.

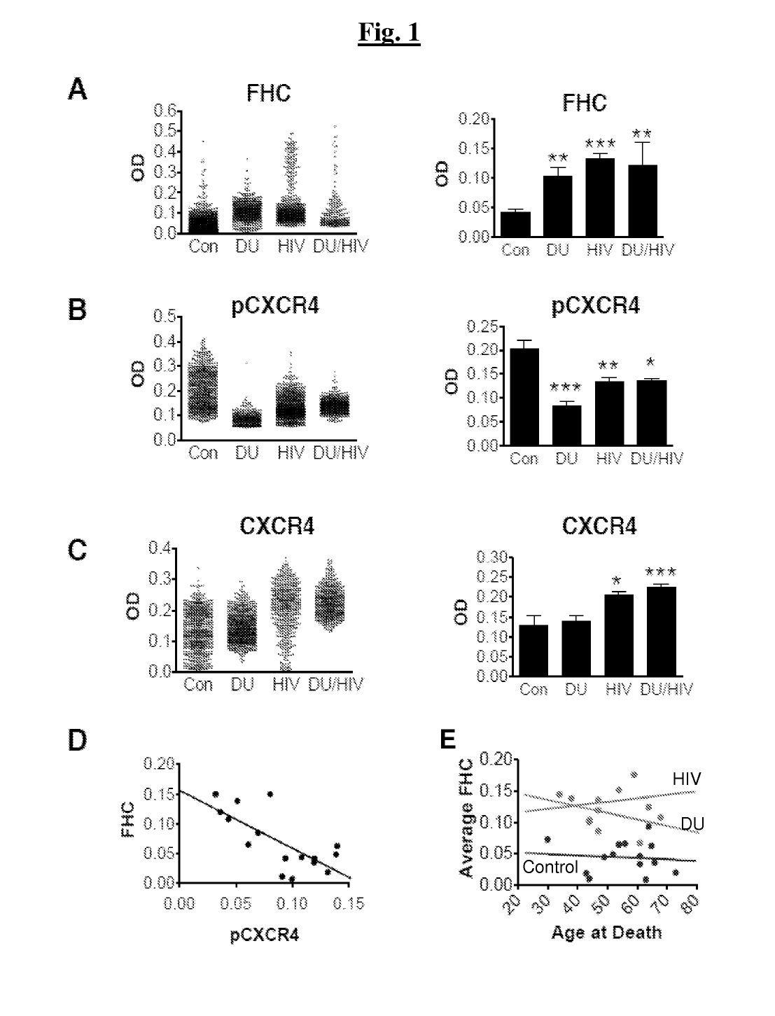

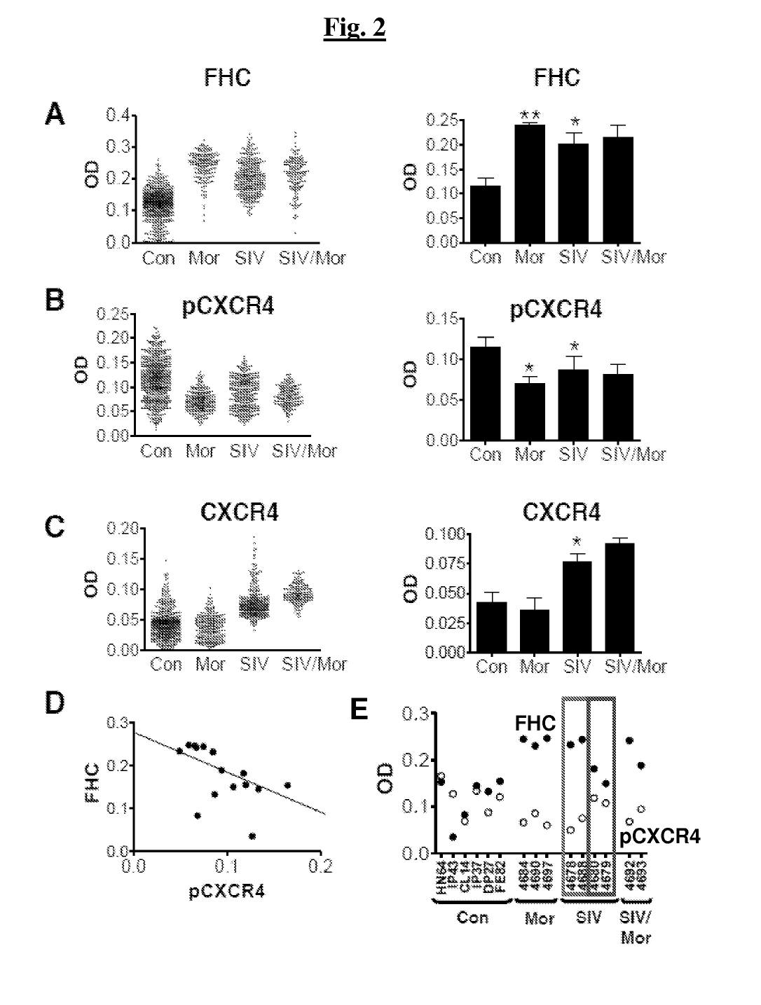

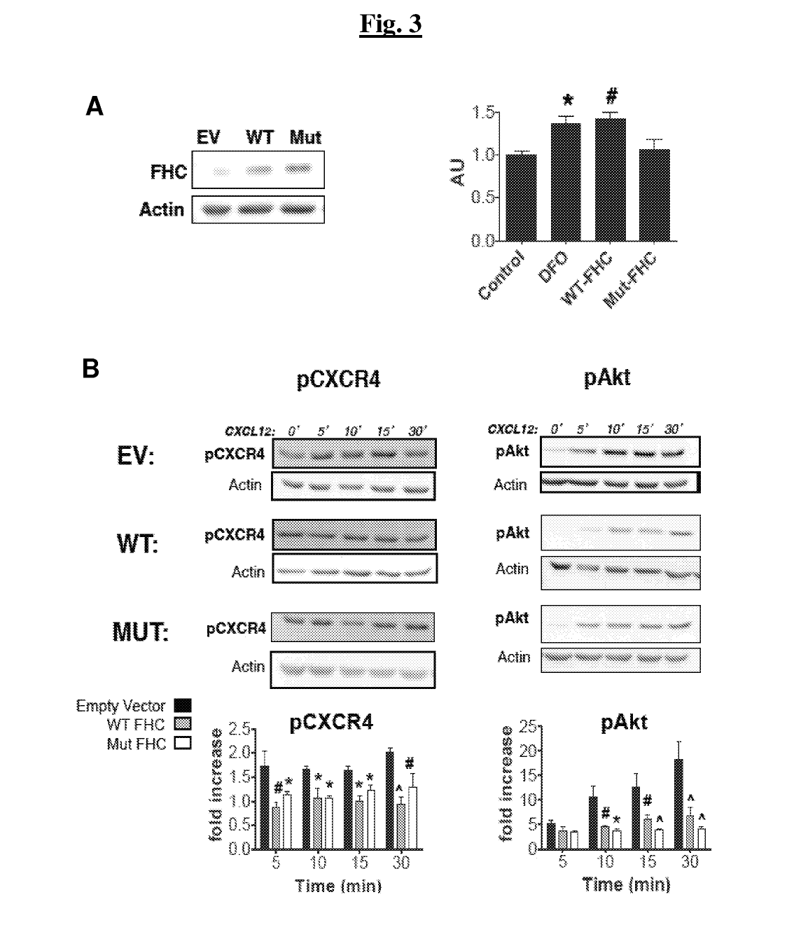

As demonstrated herein, the FHC/CXCR4 interaction is regulated by either morphine exposure or HIV infection. The combined effect of morphine and HIV/SIV infection on FHC levels was similar to each condition alone, suggesting no additive effect; the mechanisms involved in these pathways may converge or initiate feedback thereby preventing further increases in FHC. As illustrated in FIGS. 1-2, the decrease in neuronal pCXCR4 levels associated with HIV and SIV infection occurred despite increases in total CXCR4 levels, suggesting a greater degree of receptor inhibition, and a potentially additive effect of HIV infection and opiate use on CXCR4 signaling. These data also indicate important correlations between neuronal FHC levels and HAND; among HIV+ patients neuronal FHC levels correlated significantly with MSK scores, a clinical measure of cognitive impairment.

Additionally, correlations between disease severity and FHC/pCXCR4 alterations observed among SIV-infected macaques suggest that FHC alterations, which could potentially be detected in CSF or peripheral blood samples, may be a useful biomarker to assess HIV neuropathology.

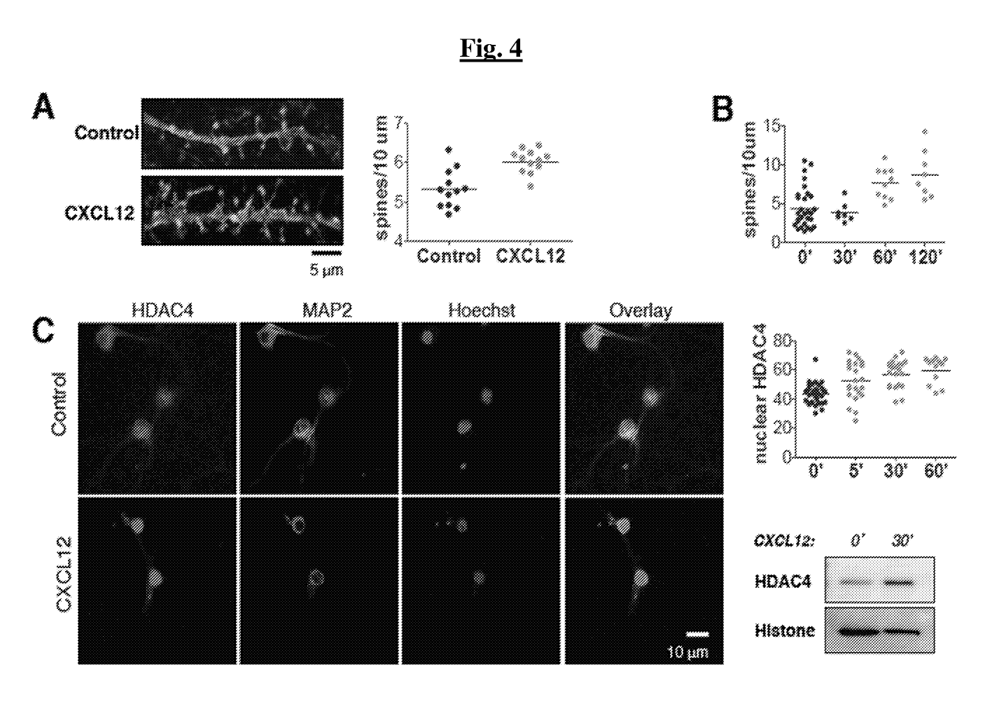

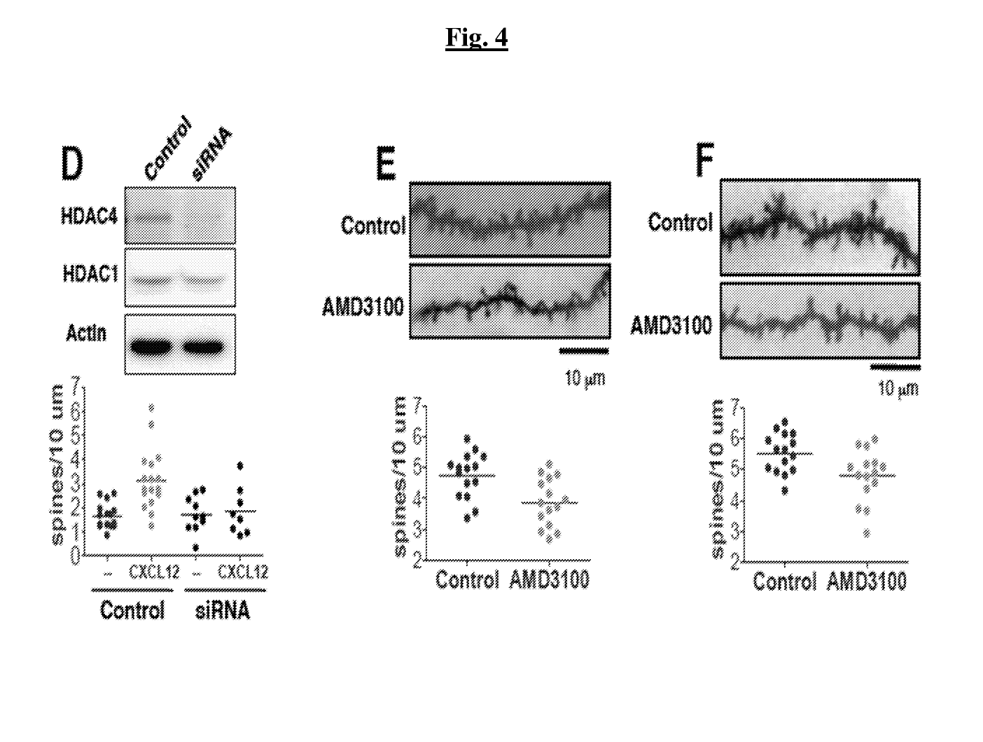

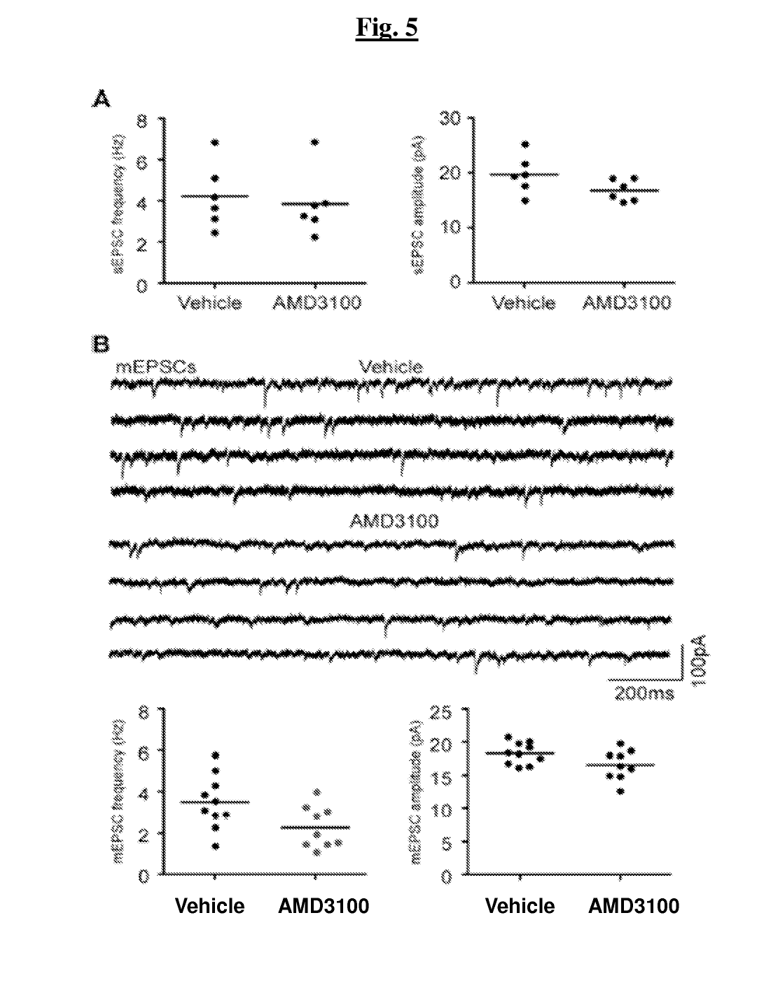

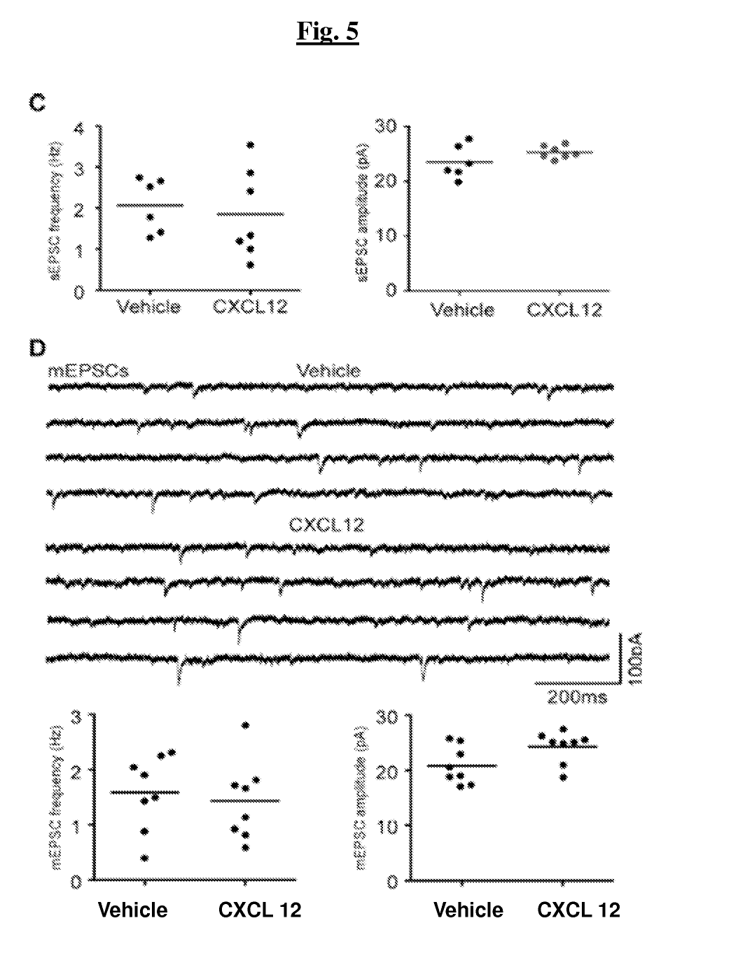

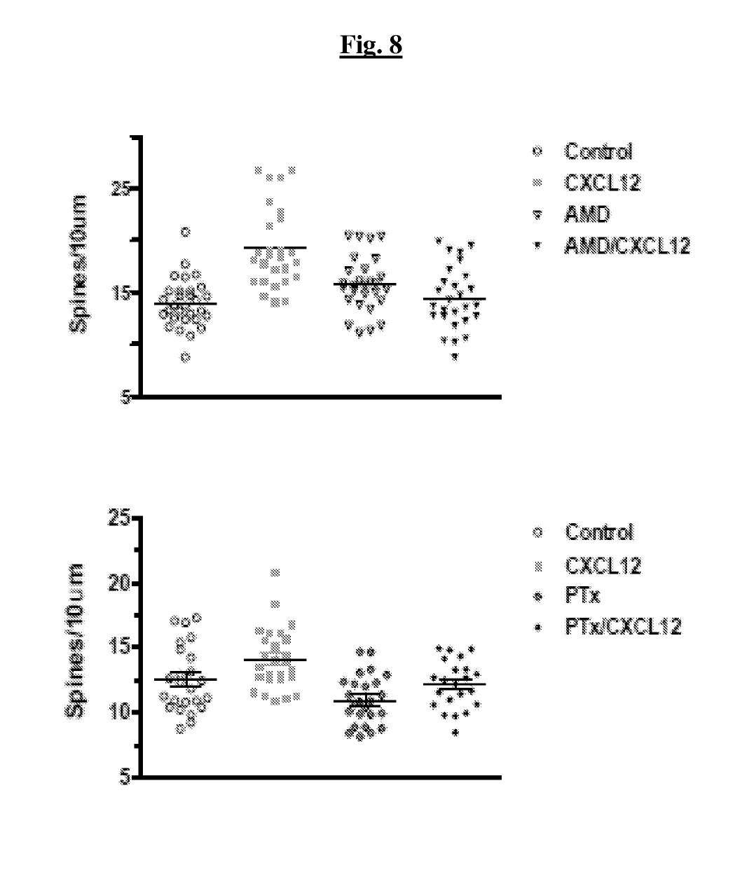

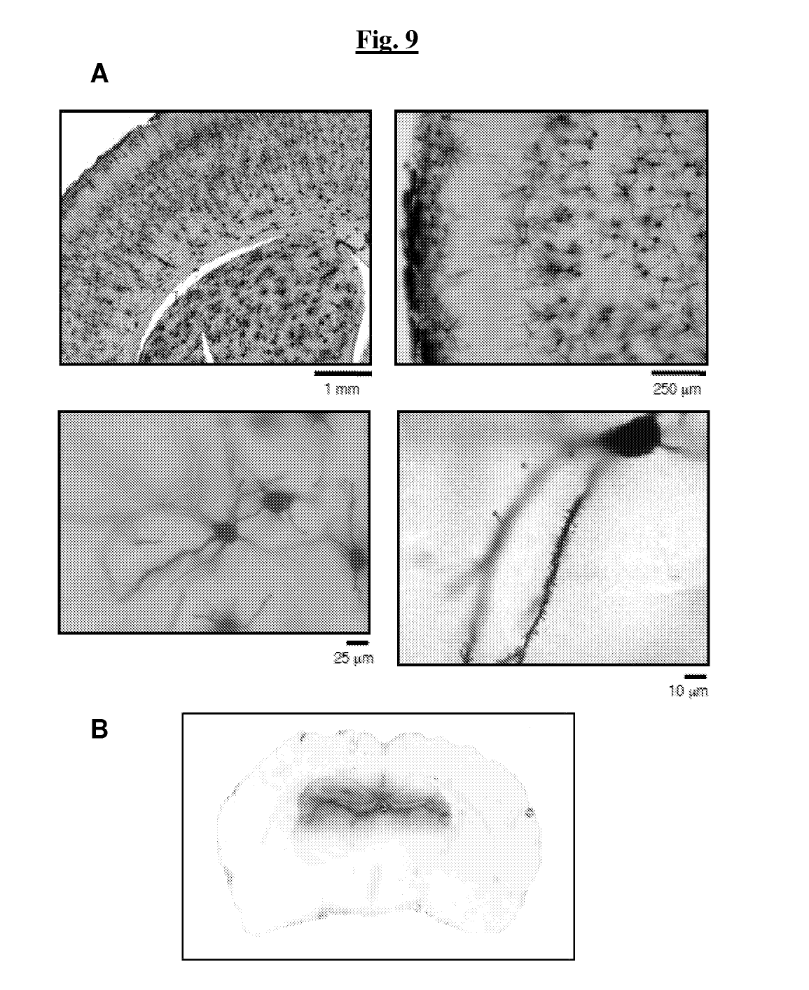

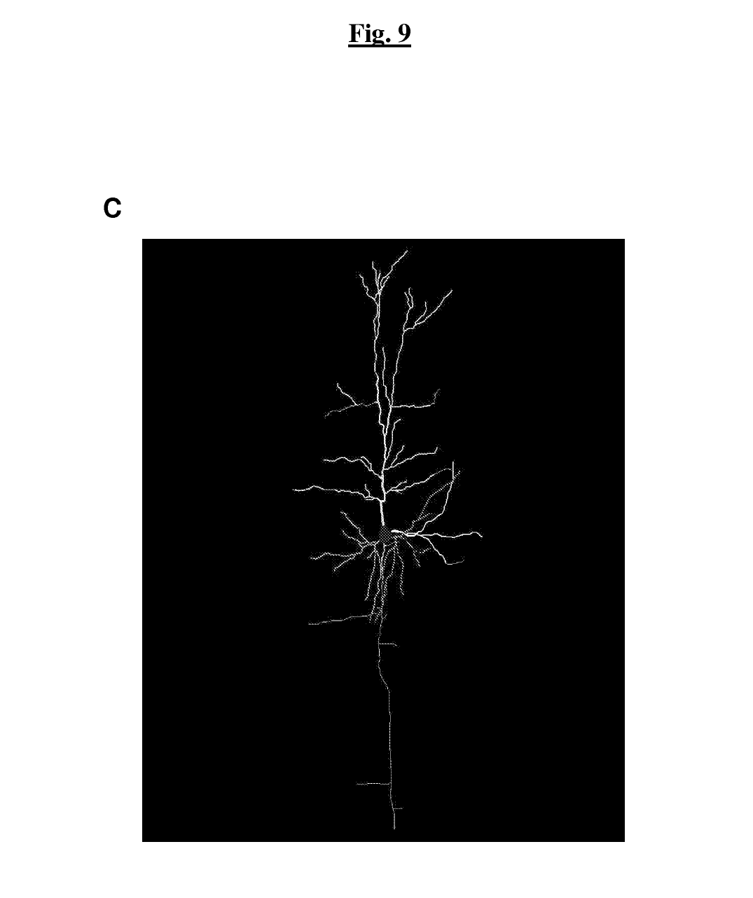

Further, FHC's CXCR4-regulatory activity was shown to be iron-independent (FIG. 3). The predominant function of this protein is to bind, oxidize, and sequester free reactive iron, and alterations of this critical activity could have severe consequences on cellular iron availability and oxidative stress. A mechanistic distinction between CXCR4 regulation and iron-binding activity of FHC thus implies the ability of targeting these functions independently; enhancing CXCR4 signaling by preventing its association with FHC should be possible without necessarily disrupting FHC's critical iron-binding functions. A possible target for specifically blocking this association may be phosphorylation of FHC at serine 178; phosphorylation at this site is essential for its association with CXCR4, and may not impact its iron-binding activity. The finding that CXCL12/CXCR4 signaling positively regulates the density of dendritic spines (FIG. 4) constitutes a novel mechanism by which this chemokine/receptor pair promotes the survival and function of mature neurons. In certain embodiments, this effect depends on G-protein mediated CXCR4 signaling (FIG. 8), HDAC4 expression (FIGS. 4C-4D) and its interaction with the transcription factor MEF2. Due to their critical roles in post-synaptic calcium buffering and synaptic plasticity, spines are essential and dynamic components of neuronal physiology. The CXCL12/CXCR4 signaling pair also decreases excitotoxicity by regulating the subunit composition of extrasynaptic NMDA receptors, and promotes tissue repair by regulating differentiation and migration of progenitor cells in models of multiple sclerosis and ischemia.

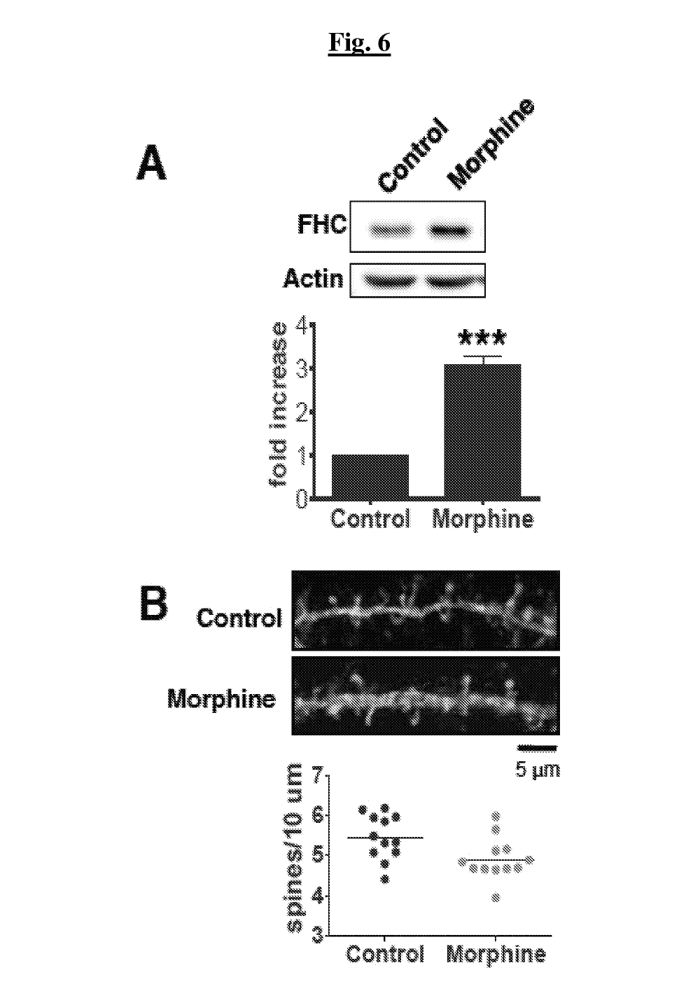

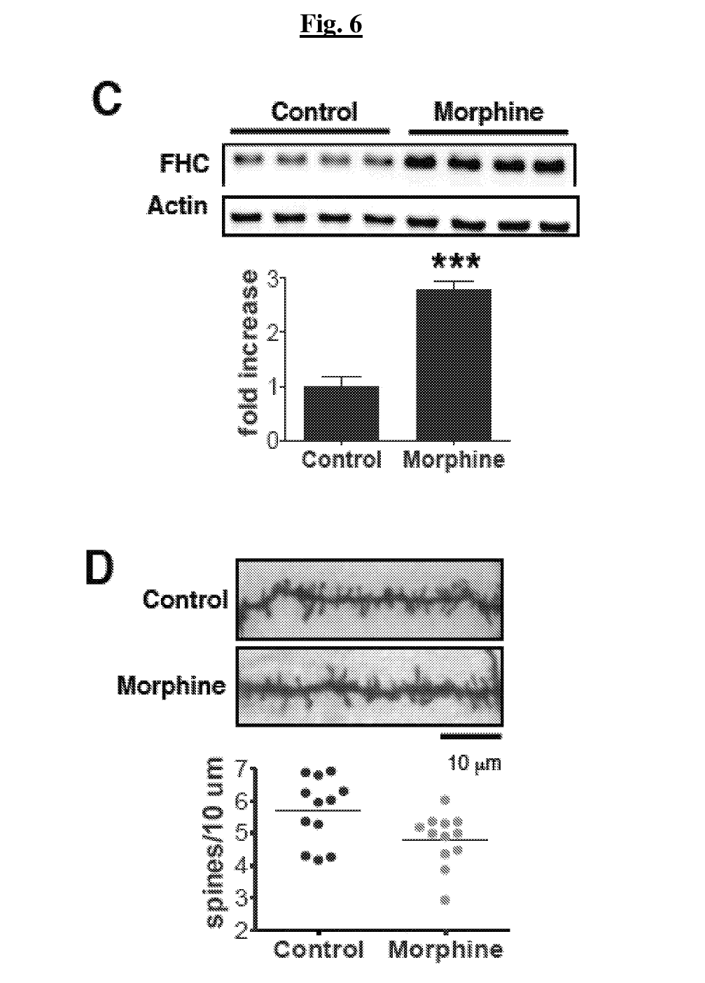

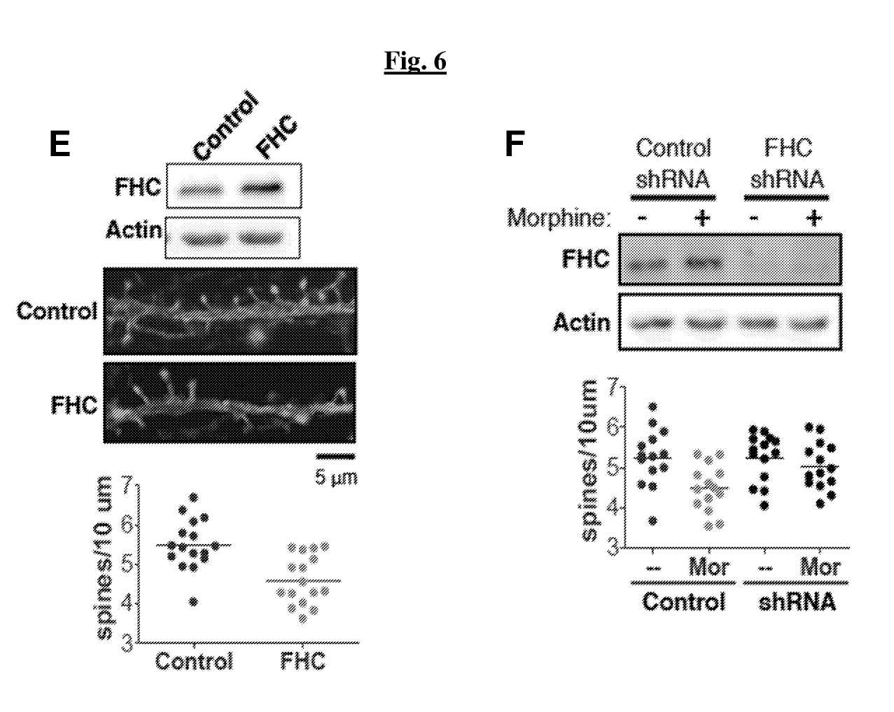

Additionally, a loss of basal CXCR4 signaling in vivo was shown to result in reduced cortical spine density. Concomitant electrophysiological alterations confirmed the functional relevance of these structural changes. In line with these data, stimulation of CXCR4 by exogenous CXCL12 increased spine density in cultured neurons. A reduction in this basal CXCR4 signaling likely underlies FHC-mediated spine loss, as observed following FHC over-expression or induction by morphine (FIG. 6). The loss of CXCR4-mediated signaling may be particularly detrimental in HIV patients, due to the reported role of specific matrix metalloproteinases (MMPs) in HIV-associated neurodegeneration. Proteolityc processing of CXCL12 by MMP2 generates a cleaved form of the chemokine (CXCL12 5-67), which is not only deficient in CXCR4 binding but also triggers neurotoxic pathways via the CXCR3 receptor. In one non-limiting embodiment, opiates exacerbate this process by influencing release of MMPs from microglia. On the other hand, the present data indicate that CXCL12-induced changes in spine density mainly depend on engagement of neuronal CXCR4, which highlights the role of the FHC/CXCR4 interaction.

The present results suggest a role for FHC in morphine-induced spine loss, identifying a pathway involved in this effect and disclosing an important contribution of constitutive CXCL12/CXCR4 signaling to basic neuronal function. The present studies support a model in which elevated neuronal FHC resulting from opiate use or HIV infection contributes to neuronal dysfunction by altering the neuroprotective actions of the CXCL12/CXCR4 axis.

In another aspect, the present studies provide new insight into the mechanism of action of established players of HIV-induced brain dysfunction, and indicate that IL-1β-targeting therapies can be used to treat these CNS disorders. Briefly, in a small animal model that mimics relevant clinical and pathological conditions of HIV+ patients on cART (e.g., low level chronic inflammation, limited expression of viral proteins in the brain, synaptodendritic injury and neurocognitive impairment) alterations of the dendritic arbor in the PFC correlate with increased levels of the protein FHC. Furthermore, gp120-treated rats showed decreased performance in an established flexible attention task that relies on intact function of the orbitofrontal and medial prefrontal cortices. These findings highlight the crucial role of the envelope protein in dendritic damage that correlates with specific behavioral abnormalities. The data also show that IL-1β plays a role for gp120-mediated upregulation of FHC, despite the increase of other inflammatory cytokines induced by the viral protein. Without wishing to be limited by any theory, IL-1β plays a necessary and sufficient role for gp120-mediated upregulation of FHC.

Increases in FHC and subsequent downregulation of CXCR4 activation have been observed in brains of HIV+ patients in the absence of drug abuse, suggesting that viral or host factors induced by HIV may also target FHC. The present studies have identified two proinflammatory cytokines, TNF-α and IL-1β, as host factors involved in this effect. TNF-α and IL-1β regulate FHC in non-neuronal cell types. TNF-α increased FHC by acting at the transcriptional level, while IL-1β, which did not affect levels of FHC mRNA in neurons, likely acts through posttranscriptional modifications. Overall, these data indicate that inflammation of the CNS, seen in HIV as well as other neuropathologies, may play a crucial role in CXCR4 dysregulation via increases in neuronal FHC.

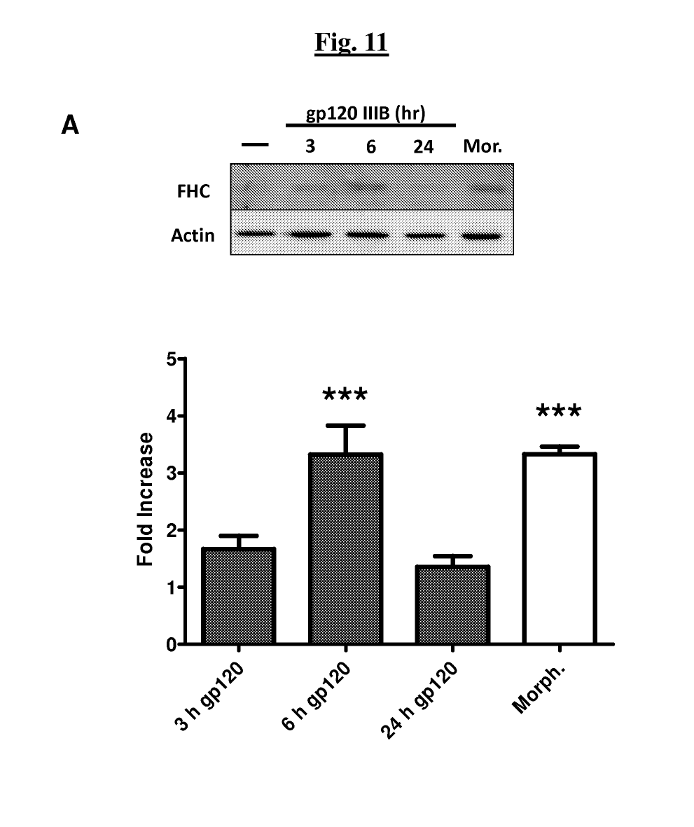

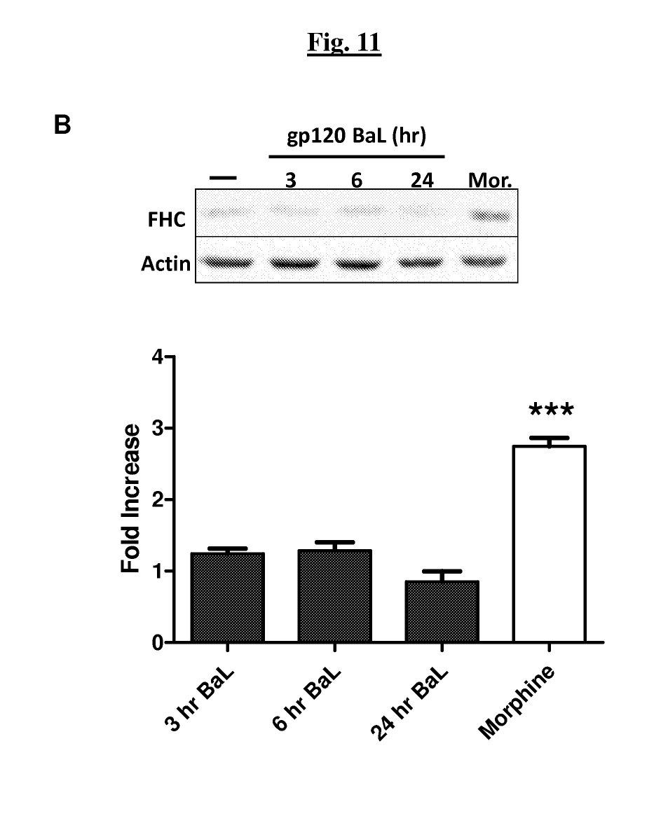

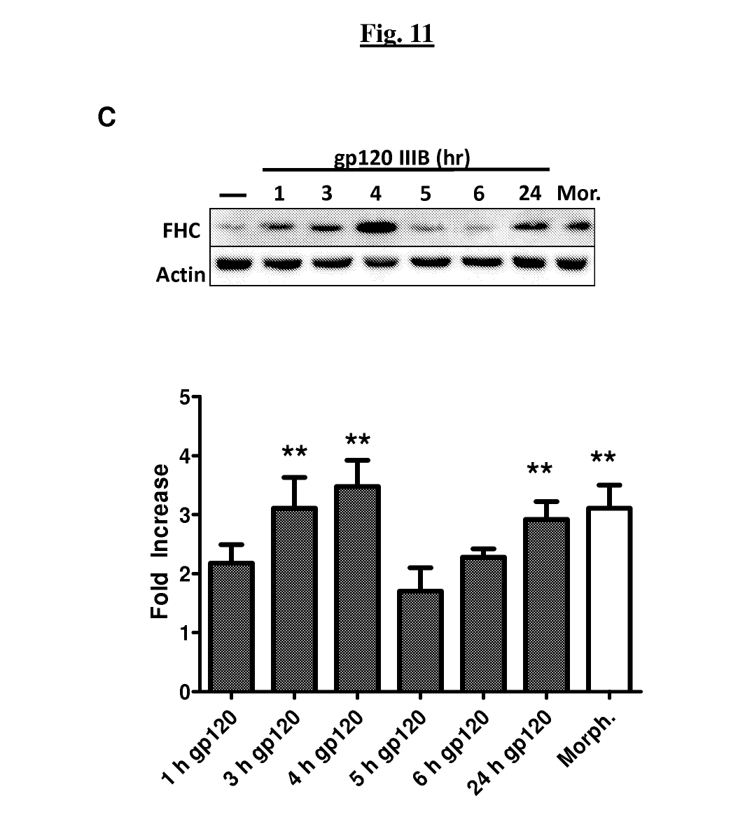

In addition to shedding light on viral-induced host factors, the present studies explored the ability of the HIV-1 envelope protein, gp120, to modulate neuronal FHC. Despite the ability of gp120_{IIIB} to directly affect neuronal signaling in neurons, the HIV protein was able to only transiently alter levels of FHC in pure neuronal cultures (FIG. 11A). This effect was not exclusive to an X4-using gp120; gp120_{BaL}, a CCR5-binding viral protein, failed to upregulate neuronal FHC in the absence of glia cells (FIG. 11B). Without wishing to be limited by any theory, the inability of either one of these envelope proteins to directly alter protein levels of FHC in neurons suggests that other intracellular mediators of gp120, such as p38 and caspase-3, are not involved in the regulation of FHC. While gp120 is able to directly bind to neurons, it also interacts with astrocytes and microglia and, in turn, induces secretion of a variety of neurotoxic substances. The presence of glia distinctly modified the ability of gp120 to upregulate neuronal FHC. Following 3, 4, or 24 hours of exposure to gp120_{IIIB}, neuronal expression of FHC protein was significantly higher compared to vehicle-treated controls. The biphasic response of FHC (FIG. 11C) may reflect the different peak secretions of TNF-α and IL-1β from glial cells, with TNF-α occurring much earlier than IL-1β. Thus, IL-1β, and not TNF-α, is responsible for gp120-mediated upregulation of FHC, despite both cytokines being secreted by the viral protein as confirmed by ELISA.

The ex vivo studies further highlighted the ability of HIV proteins to downregulate dendritic spines. Using the non-infectious HIV-1 Tg rats, a significant reduction in dendritic spine density and branching in neurons of the prefrontal cortex, compared to control animals, was observed. Additionally, levels of FHC were increased in whole brain lysates from these HIV-1 Tg rats, confirming the correlation between FHC and spine numbers. The present study thus demonstrates spine reduction in the medial PFC of HIV-1 Tg rat—a crucial finding, since dendritic spine density is one of the pathophysiological changes well correlated with cognitive impairment in HIV patients. The animals of the present study also showed behavioral changes consistent with such deficit. Despite the physical limitations of the HIV-1 Tg rat (i.e., congenital cataracts), neurological deficits reminiscent of the human condition are observed in these animals.

The investigations in rats treated with gp120 shed further light on factor(s) involved in the neurocognitive impairment. The behavioral analyses performed indicated that specific aspects of cognitive flexibility, including reversal learning and extradimensional shift, were altered in animals whose brain tissue was exposed to low concentrations of gp120 for a prolonged period of time. The negative outcome of experiments with Fischer 344 animals is suggestive of gp120-induced impairment of cognitive function in these animals. The results from gp120-treated SD rats on the other hand indicated deficits in flexible attention, a cognitive ability dependent on intact prefrontal cortical function. Layer II/III pyramidal neurons from gp120-treated rats also had fewer spines compared to control animals. Although these results do not exclude the possible contribution of other viral proteins, they provide evidence about the ability of gp120 treatment to reduce spine and branch number in vivo and affect executive function.

Outside of spine numbers, additional analysis of dendritic spines must be considered, including spine type and location. Spines are generally categorized as either mushroom or thin, with other shapes existing, such as branched or filopodial. Mushroom spines are thought to be more stable and mature, with larger post-synaptic densities and more AMPA glutamate receptors, while thin spines have a much higher turnover rate and are strongly correlated with the ability to learn new information. In certain embodiments, thin spines in gp120-treated rats are preferentially downregulated compared to mushroom spines.

The present study bears important implications for understanding mechanisms of dendritic spine loss in the HIV-infected brain by focusing on potential contributors to neurocognitive impairment in the post-cART era, where productive viral replication is limited.

The lack of the infectious component in both the in vitro and in vivo models used here allowed the identification of specific viral (i.e. gp120) and host (IL1β/TNF-α) factors with intrinsic ability to alter FHC independently of other co-morbid factors (i.e., drug abuse, aging, altered iron metabolism) or overt inflammation. The ability of FHC to modulate CXCR4 signaling is outside of its critical iron-sequestration and storage functions, suggesting that its association to CXCR4 can be specifically targeted without detrimental effects to the cell.

Compounds and/or Compositions

In one aspect, the invention includes a composition that down-regulates FHC in a subject. In yet another aspect, the invention includes a composition that down-regulates (i.e., decreases the concentration, expression level and/or activity of) IL-1β in a subject. In certain embodiments, the subject is a mammal. In other embodiments, the mammal is human. For example and without limitation, the composition may comprise at least one selected from the group consisting of a small interfering RNA (siRNA), a microRNA, an antisense nucleic acid, a ribozyme, an expression vector encoding a transdominant negative mutant, an intracellular antibody, a peptide and a small molecule compound.

In certain embodiments, the compositions of the invention include interleukin-1 receptor antagonist (IL-1ra), anakinra (KINERET™) and/or rilonacept (ARCALYST™). IL-1ra is an agent that binds non-productively to the cell surface interleukin-1 receptor (IL-1R), which is the same receptor that binds IL-1. IL-1ra thus prevents IL-1 from sending a signal to that cell. Anakinra and rilonacept are large recombinant proteins that directly target interleukin 1 (IL-1) signaling, by either interfering with the IL-1 receptor (IL-1R) or sequestering IL-1 from the circulation. Anakinra is a human IL-1R antagonist, thus binding to IL-1R and blocking its binding of IL-1α or IL-1β. Rilonacept is a dimeric protein comprising the ligand-binding domains of the extracellular portions of the human IL-1R and human IL-1R accessory protein linked to the Fc portion of human immunoglobulin G1 (IgG1). Rilonacept binds and neutralizes circulating IL-1.

One skilled in the art will appreciate, based on the disclosure provided herein, that one way to decrease the mRNA and/or protein levels of FHC and/or IL-1β in a cell is by reducing or inhibiting expression of the nucleic acid encoding FHC and/or IL-1β. Thus, the protein level of FHC and/or IL-1β in a cell may also be decreased using a molecule or compound that down-regulates FHC and/or IL-1β gene expression such as, for example, an antisense molecule or a ribozyme.

By way of a non-limited example, down-regulation of FHC and/or IL-1β is described below in the context of decreasing the mRNA and/or protein level of FHC and/or IL-1β in a cell by reducing or inhibiting expression of the nucleic acid encoding FHC and/or IL-1β.

In certain embodiments, the modulating sequence is an antisense nucleic acid sequence that is expressed by a plasmid vector. The antisense expressing vector is used to transfect a mammalian cell or the mammal itself, thereby causing reduced endogenous expression of FHC and/or IL-1β. However, the invention should not be construed to be limited to inhibiting expression of FHC and/or IL-1β by transfection of cells with antisense molecules. Rather, the invention encompasses other methods known in the art for inhibiting expression or activity of FHC and/or IL-1β including, but not limited to, the use of a ribozyme, the expression of a non-functional component of FHC and/or IL-1β (i.e., transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific Amerimay 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (Anal. Biochem. 1988, 172:289). Such antisense molecules may be provided to the cell via genetic expression using DNA encoding the antisense molecule as taught by U.S. Pat. No. 5,190,931 by Inoue.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

The ability to specifically inhibit gene function in a variety of organisms utilizing antisense RNA or dsRNA-mediated interference (RNAi or dsRNA) is well known in the fields of molecular biology. dsRNA (RNAi) typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules and should not hybridize with sequences within the hybridizing portions of the dsRNA molecule. The specificity of this gene silencing mechanism appears to be extremely high, blocking expression only of targeted genes, while leaving other genes unaffected. Accordingly, one method for treating retroviral infection according to the invention comprises the use of materials and methods utilizing double-stranded interfering RNA (dsRNAi), or RNA-mediated interference (RNAi) comprising polynucleotide sequences identical or homologous to a desired component of TGF-β signaling pathway. The terms “dsRNAi”, “RNAi”, “iRNA”, and “siRNA” are used interchangeably herein unless otherwise noted.

RNA containing a nucleotide sequence identical to a fragment of the target gene is preferred for inhibition; however, RNA sequences with insertions, deletions, and point mutations relative to the target sequence may also be used for inhibition. Sequence identity may optimized by sequence comparison and alignment algorithms known in the art (Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase may be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see, for example, International Application No. WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA may be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Fragments of genes may also be utilized for targeted suppression of gene expression. These fragments are typically in the approximate size range of about 20 consecutive nucleotides of a target sequence. Thus, targeted fragments are preferably at least about 15 consecutive nucleotides. In certain embodiments, the gene fragment targeted by the RNAi molecule is about 20-25 consecutive nucleotides in length. In a more preferred embodiment, the gene fragments are at least about 25 consecutive nucleotides in length. In an even more preferred embodiment, the gene fragments are at least 50 consecutive nucleotides in length. Various embodiments also allow for the joining of one or more gene fragments of at least about 15 nucleotides via linkers. Thus, RNAi molecules useful in the practice of the instant invention may contain any number of gene fragments joined by linker sequences.

In certain embodiments, the invention includes full length or fragments of FHC and/or IL-1β. The gene fragments may range from one nucleotide less than the full-length gene. Nucleotide sequences for FHC and/or IL-1β are known in the art and may be obtained from patent publications, public databases containing nucleic acid sequences, or commercial vendors. A skilled artisan would understand that the disclosure presented herein provides sufficient written support for any fragment length ranging from about 15 consecutive polynucleotides to one nucleotide less than the full length polynucleotide sequence of FHC and/or IL-1β may have a whole number value ranging from about 15 consecutive nucleotides to one nucleotide less than the full length polynucleotide.

Accordingly, methods utilizing RNAi molecules in the practice of the invention are not limited to those that are targeted to the full-length polynucleotide or gene. Gene product may be inhibited with an RNAi molecule that is targeted to a portion or fragment of the exemplified polynucleotides; high homology (90-95%) or greater identity is also preferred, but not essential, for such applications.

In another aspect of the invention, the dsRNA molecules of the invention may be introduced into cells with single stranded (ss) RNA molecules that are sense or anti-sense RNA derived from the nucleotide sequences disclosed herein. Methods of introducing ssRNA and dsRNA molecules into cells are well-known to the skilled artisan and includes transcription of plasmids, vectors, or genetic constructs encoding the ssRNA or dsRNA molecules according to this aspect of the invention; electroporation, biolistics, or other well-known methods of introducing nucleic acids into cells may also be used to introduce the ssRNA and dsRNA molecules of this invention into cells.

Other types of gene inhibition technology may be used to down-regulate FHC and/or IL-1β in a cell. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules may be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences that are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of FHC and/or IL-1β may be designed by incorporating target sequences into the basic ribozyme structure that are complementary to the mRNA sequence of FHC and/or IL-1β. Ribozymes targeting FHC and/or IL-1β may be synthesized using commercially available reagents (Applied Biosystems, Inc., Foster City, Calif.) or they may be genetically expressed from DNA encoding them.

In another aspect of the invention, FHC and/or IL-1β may be down-regulated by way of inactivating and/or sequestering FHC and/or IL-1β. As such, down-regulating FHC and/or IL-1β may be accomplished by using a transdominant negative mutant. Alternatively an intracellular antibody specific for FHC and/or IL-1β, otherwise known as an antagonist to FHC and/or IL-1β, may be used. In certain embodiments, the antagonist is a protein and/or compound having the desirable property of interacting with a binding partner of FHC and/or IL-1β and thereby competing with FHC and/or IL-1β. In other embodiments, the antagonist is a protein and/or compound having the desirable property of interacting with FHC and/or IL-1β and thereby sequestering FHC and/or IL-1β.

By way of a non-limited example, an antibody is described below as an example of inactivating and/or sequestering FHC and/or IL-1β.

As will be understood by one skilled in the art, any antibody that may recognize and specifically bind to FHC and/or IL-1β is useful in the present invention. The invention should not be construed to be limited to any one type of antibody, either known or heretofore unknown, provided that the antibody may specifically bind to FHC and/or IL-1β. Methods of making and using such antibodies are well known in the art. For example, the generation of polyclonal antibodies may be accomplished by inoculating the desired animal with the antigen and isolating antibodies which specifically bind the antigen therefrom. Monoclonal antibodies directed against full length or peptide fragments of a protein or peptide may be prepared using any well known monoclonal antibody preparation procedures, such as those described, for example, in Harlow et al. (1989, Antibodies, A Laboratory Manual, Cold Spring Harbor, N.Y.) and in Tuszynski et al. (1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. However, the invention should not be construed as being limited solely to methods and compositions including these antibodies, but should be construed to include other antibodies, as that term is defined elsewhere herein.

In some instances, it is desirable to prepare monoclonal antibodies from various mammalian hosts, such as rodents (e.g., mice), primates (e.g., humans), etc. Descriptions of techniques for preparing such monoclonal antibodies are well known and are described, for example, in Harlow et al., ANTIBODIES: A LABORATORY MANUAL, COLD SPRING HARBOR LABORATORY, Cold Spring Harbor, N.Y. (1988); Harlow et al., USING ANTIBODIES: A LABORATORY MANUAL, (Cold Spring Harbor Press, New York, 1998); Breitling et al., RECOMBINANT ANTIBODIES (Wiley-Spektrum, 1999); and Kohler et al., 1997 Nature 256: 495-497; U.S. Pat. Nos. 5,693,762; 5,693,761; 5,585,089; and 6,180,370.

Nucleic acid encoding an antibody obtained using the procedures described herein may be cloned and sequenced using technology that is available in the art, and is described, for example, in Wright et al. (Critical Rev. in Immunol. 1992, 12:125-168) and the references cited therein. Further, the antibody of the invention may be “humanized” using the technology described in Wright et al. (supra) and in the references cited therein, and in Gu et al. (Thrombosis and Hematocyst 1997, 77:755-759).

Alternatively, antibodies may be generated using phage display technology. To generate a phage antibody library, a cDNA library is first obtained from mRNA that is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, New York).

Bacteriophage that encode the desired antibody may be engineered such that the protein is displayed on the surface thereof in such a manner that it is available for binding to its corresponding binding protein, e.g., the antigen against which the antibody is directed. Thus, when bacteriophage that express a specific antibody are incubated in the presence of a cell that expresses the corresponding antigen, the bacteriophage will bind to the cell. Bacteriophage that do not express the antibody will not bind to the cell. Such panning techniques are well known in the art and are described for example, in Wright et al. (Critical Rev. in Immunol. 1992, 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage that encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J Mol Biol 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

The invention encompasses polyclonal, monoclonal, synthetic antibodies, and the like. One skilled in the art would understand, based upon the disclosure provided herein, that the crucial feature of the antibody of the invention is that the antibody specifically bind with FHC and/or IL-1β.

In other related aspects, the invention includes an isolated nucleic acid encoding a down-regulator of the invention, operably linked to a nucleic acid comprising a promoter/regulatory sequence such that the nucleic acid is preferably capable of directing expression of the down-regulator encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

In another aspect, the invention includes a vector comprising an siRNA polynucleotide. Preferably, the siRNA polynucleotide is capable of inhibiting the expression of a target polypeptide, wherein the target polypeptide is FHC and/or IL-1β. The incorporation of a desired polynucleotide into a vector and the choice of vectors is well-known in the art as described in, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems may be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. See, e.g., International Applications Nos. WO 01/96584 and WO 01/29058; and U.S. Pat. No. 6,326,193.

For expression of the desired down-regulator of FHC and/or IL-1β, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements may be increased to 50 bp apart before activity begins to decline. Depending on the promoter, it appears that individual elements may function either co-operatively or independently to activate transcription.

A promoter may be one naturally associated with a gene or polynucleotide sequence, as may be obtained by isolating the 5′ non-coding sequences located upstream of the coding segment and/or exon. Such a promoter may be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, may be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001, Molecular Cloning: A Laboratory Manual, Cold Spring Harbor Laboratory, New York). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the desired down-regulator of FHC and/or IL-1β, the expression vector to be introduced into a cell may also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector may be readily introduced into a host cell, e.g., mammalian, bacterial, yeast or insect cell by any method in the art. For example, the expression vector may be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors may be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the down-regulator of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To generate a genetically modified cell, any DNA vector or delivery vehicle may be utilized to transfer the desired FHC and/or IL-1β down-regulator to a cell in vitro or in vivo. In the case where a non-viral delivery system is utilized, a preferred delivery vehicle is a liposome. The above-mentioned delivery systems and protocols therefore may be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

In addition to a genetic approach, the invention includes the use of small molecule compounds to down-regulate FHC and/or IL-1β. Without wishing to be limited by theory, intracellular iron levels may regulate FHC and/or IL-1β expression (e.g., FHC and/or IL-1β translation is inhibited when iron levels are low). In certain embodiments, the small molecule compound includes an iron chelating agent, such as but not limited to, EDTA (also known as ethylenediaminetetraacetic acid), EDDHA (also known as ethylenediamine-N,N′-bis(2-hydroxyphenylacetic acid), deferoxamine (also known as desferrioxamine B, desferoxamine B, DFO-B, DFOA, DFB, desferal, N′-{5-[acetyl(hydroxy)amino]pentyl}-N-[5-({4-[(5-aminopentyl) (hydroxy)amino]-4-oxobutanoyl}amino)pentyl]-N-hydroxysuccinamide, or N′-[5-(acetyl-hydroxy-amino)pentyl]-N-[5-[3-(5-aminopentyl-hydroxy-carbamoyl) propanoylamino]pentyl]-N-hydroxy-butane diamide), deferasinox (also known as [4-[(3Z,5E)-3,5-bis(6-oxo-1-cyclohexa-2,4-dienylidene)-1,2,4-triazolidin-1-yl]benzoic acid), or deferiprone (also known as 3-hydroxy-1,2-dimethylpyridin-4(1H)-one).

In one aspect, the invention includes a composition that up-regulates FHC and/or IL-1β in a subject. In certain embodiments, the subject is a mammal. In other embodiments, the mammal is human. For example and without limitation, the composition may comprise at least one selected from the group consisting of a small interfering RNA (siRNA), a microRNA, an antisense nucleic acid, a ribozyme, an expression vector encoding a transdominant negative mutant, an intracellular antibody, a peptide and a small molecule compound.

In certain embodiments, the composition comprises an opioid agonist or a salt thereof. In other embodiments, the opioid agonist is at least one selected from the group consisting of adrenorphin, amidorphin, casomorphin, DADLE (also known as [D-Ala², D-Leu⁵]-Enkephalin; L-Tyrosyl-D-alanylglycyl-L-phenylalanyl-D-Leucine; N—(N—(N—(N-L-tyrosyl-D-alanyl)glycyl)-L-phenylalanyl)-D-Leucine; or Tyr-D-Ala-Gly-Phe-D-Leu-OH), DAMGO, dermorphin, endomorphin, morphiceptin, octreotide, opiorphin, TRIMU 5 (also known as L-tyrosyl-N-{[(3-methylbutyl)amino]acetyl}-D-alaninamide, or (2S)-2-amino-3-(4-hydroxyphenyl)-N-[(2R)-1-{[2-(3-methylbutylamino)acetyl]amino}-1-oxopropan-2-yl]propanamide), codeine, morphine, thebaine, oripavine, esters of morphine (such as but not limited to diacetylmorphine, nicomorphine, dipropanoylmorphine, diacetyldihydromorphine, acetylpropionylmorphine, desomorphine, methyldesorphine, and dibenzoylmorphine), ethers of morphine (such as but not limited to dihydrocodeine, ethylmorphine, and heterocodeine), semi-synthetic alkaloid derivatives (such as but not limited to buprenorphine, etorphine, hydrocodone, hydromorphone, oxycodone, and oxymorphone), anilidopiperidines (such as but not limited to fentanyl, alphamethylfentanyl, alfentanil, sufentanil, remifentanil, carfentanyl, and ohmefentanyl), phenylpiperidines (such as but not limited to pethidine (meperidine), ketobemidone, MPPP, allylprodine, prodine, and PEPAP), diphenylpropylamine derivatives (such as but not limited to propoxyphene, dextropropoxyphene, dextromoramide, bezitramide, piritramide, methadone, dipipanone, levomethadyl acetate (LAAM), difenoxin, diphenoxylate and loperamide), benzomorphan derivatives (such as but not limited to phenazocine), oripavine derivatives (such as but not limited to buprenorphine, dihydroetorphine and etorphine), morphinan derivatives (such as but not limited to levorphanol and levomethorphan), lefetamine, menthol, meptazinol, mitragynine, tilidine, tramadol and tapentadol.

The compounds included in the compositions of the invention may form salts with acids, and such salts are included in the present invention. In certain embodiments, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

### Methods

In one aspect, the invention includes a method of treating or preventing HAND in a subject in need thereof. In certain embodiments, the method comprises administering to the subject a therapeutically effective amount of a pharmaceutical composition that down-regulates FHC in the subject, whereby HAND is treated or prevented in the subject. In other embodiments, the method comprises administering to the subject a therapeutically effective amount of a pharmaceutical composition that decreases the concentration, expression level and/or activity of IL-1β in the subject, whereby HAND is treated or prevented in the subject.

In another aspect, the invention includes a method of treating or preventing in a subject in need thereof a disease or condition associated with CXCR4 up-regulation. In certain embodiments, the method comprises administering to the subject a therapeutically effective amount of a pharmaceutical composition comprising an opioid agonist, whereby the disease or condition is treated or prevented.

In certain embodiments, the composition comprises an agent selected from the group consisting of an antibody, siRNA, ribozyme, antisense, aptamer, peptidomimetic, iron chelating agent, and combinations thereof. In other embodiments, the antibody comprises an antibody selected from a polyclonal antibody, monoclonal antibody, humanized antibody, synthetic antibody, heavy chain antibody, human antibody, biologically active fragment of an antibody, and combinations thereof. In yet other embodiments, the subject is a mammal. In yet other embodiments, the mammal is human. In yet other embodiments, the composition is administered by an inhalational, oral, rectal, vaginal, parenteral, intracranial, topical, transdermal, pulmonary, intranasal, buccal, ophthalmic, intrathecal, or intravenous route of administration.

In certain embodiments, the subject is further administered at least one anti-HIV drug. In other embodiments, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

In certain embodiments, the disease or condition comprises cancer, metastasis, liver fibrosis, HIV infection or pain. In other embodiments, liver fibrosis includes HIV-associated liver fibrosis.

In certain embodiments, the opioid agonist is at least one selected from the group consisting of adrenorphin, amidorphin, casomorphin, DADLE, DAMGO, dermorphin, endomorphin, morphiceptin, octreotide, opiorphin, TRIMU 5, codeine, morphine, thebaine, oripavine, esters of morphine, ethers of morphine, semi-synthetic alkaloid derivatives, anilidopiperidines, phenylpiperidines, diphenylpropylamine derivatives, benzomorphan derivatives, oripavine derivatives, morphinan derivatives, lefetamine, menthol, meptazinol, mitragynine, tilidine, tramadol and tapentadol.

In certain embodiments, the disease or condition comprises HIV infection and the subject is further administered at least one anti-HIV drug.

### Combination Therapies

The compounds and compositions identified using the methods described here are useful in the methods of the invention in combination with one or more additional compounds useful for treating the diseases or disorders contemplated herein. These additional compounds may comprise compounds identified herein or compounds, e.g., commercially available compounds, known to treat, prevent, or reduce the symptoms of the diseases or disorders contemplated herein.

In non-limiting examples, the compounds and compositions useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entiy and Fusion Inhibitors: maraviroc (Celsentri®, Selzentiy®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddl, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention also encompasses the use of pharmaceutical compositions of the invention to practice the methods of the invention.

Such pharmaceutical compositions may be provided in a form suitable for administration to a subject, and may comprise one or more pharmaceutically acceptable carriers, one or more additional ingredients, or some combination of these. The compositions of the invention may comprise a physiologically acceptable salt, such as a compound contemplated within the invention in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, intracranial, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 g to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition. For example, In certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, intracranial, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Additional Administration Forms Additional dosage forms of this invention include dosage forms as described in U.S. Pat. Nos. 6,340,475, 6,488,962, 6,451,808, 5,972,389, 5,582,837, and 5,007,790.

Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

Controlled Release Formulations and Drug Delivery Systems Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials & Methods:

Unless otherwise noted, all starting materials and resins were obtained from commercial suppliers and used without purification.

Cell culture media were purchased from Invitrogen, and Holtzman rats for both cell culture and in vivo experiments were obtained from Harlan. Morphine sulfate, DFO (deferoxamine), AMD3100, and pertussis toxin were purchased from Sigma Aldrich, and recombinant CXCL12 from Peprotech. Morphine pellets were obtained from NIDA, and mutant/wild type FHC plasmids a gift from Dr. Paolo Arosio (University of Brescia, Italy).

### Statistics:

Data are represented as mean±SEM. Both human and non-human primate databases were generated using the SPSS statistical package (IBM). The statistics for mean differences in optical densities between human disease groups were calculated by one-way ANOVA followed by Bonferroni's multiple comparison. Rhesus macaque treatment groups were analyzed with an unpaired 2-tailed T test. Graphpad prism statistical software was used for these analyses and a P value less than 0.05 considered significant. The correlation between FHC and pCXCR4 optical densities (FIG. 1D) and average FHC OD and Age at Death (FIG. 1E) were calculated using a Pearson correlation on Graphpad prism and SPSS, respectively. Western blot densitometry and dendritic spine densities were compared by one-way ANOVA, followed by Newman-Keuls post hoc test—unless otherwise indicated.

### Human Population:

Postmortem human brain tissue was acquired through the National NeuroAIDS Tissue Consortium (NNTC). The human population studied (total N=51) was divided among individuals with a history of drug abuse (including opiate abuse) and/or documented HIV, with a degree of HIV-associated neurological impairment ranging from normal (MSK=0) to profoundly impaired (MSK=4) (Tables 2-3).

The tissue acquisition protocol may be outlined as follows (morgello et al., 2001, Neuropathol. Appl. Neurobiol. 27:326-335). Briefly, within the frontal cortex, Broadman area 8 and underlying white matter was removed at autopsy. Patient information was collected regarding HIV status, neurocognitive impairment and relevant clinical measures pertinent to disease progression and co-morbidities. The following demographic and clinical information was obtained: age at death, race, sex, HIV status, CD4+ count, CD8+ cell count, peripheral blood viral load, cerebrospinal fluid (CSF) viral load, neurocognitive impairment and drug abuse history (opiates, cocaine, stimulants, cannabis, methamphetamine, sedatives, hallucinogens and alcohol). Subjects were studied longitudinally with neurologic, psychiatric, and neurocognitive evaluations at 6 month time intervals. Psychiatric disorders were determined in the context of underlying substance abuse using the Psychiatric Research Interview for Substance and Mental Disorders (PRISM) scale (Hasin et al., 1996, Am. J. Psychiatry 153:1195-1201). The majority of patients were undergoing cART regiments and only a small number were on a structured treatment interruption, or cART naïve. Additional control samples were obtained from the DUCOM tissue procurement facility. All subjects were required to sign informed consent forms.

### Non-Human Primate Population:

Twenty Indian rhesus macaques, housed and treated at BIOQUAL, Inc (Rockville, Md.) were studied in conjunction with the human cohort to determine the effects of a fixed dosage regimen of IV morphine with and without concurrent SIV infection (Table 4). Following quarantine, all animals received a single 0.5 mL tetanus vaccine and quarterly TB tests. Tuberculosis tests were given three times, bi-weekly, accompanied with doses of praziquantel and ivermectin for deworming. Animals were given ad libidum monkey chow (Harlan Tekland chow #8714) supplemented with fruit, vegetables and feeding devices consisting of peanut butter and cereals. All treatment group animals received 5 Prima Treats to supplement dietary intake. Treatment groups included: control (N=1), SIV only (N=4), morphine only (N=3) or both SIV inoculated and morphine administered (N=2). The neurovirulent SIV viral strain, SIVMac251 was diluted in 0.9% NaCl and administered intravenously through the saphenous vein, at a final dose of 5 mg/kg. Morphine treatment began with a dose of 3 mg/kg body weight, administered 3 times daily for 2 weeks. This dose was increased to 5 mg/kg administered 3 times daily for the remaining time (total treatment time was 90-110 days). Animals were then sacrificed, and the frontal cortex was removed, fixed in formalin, and paraffin-embedded for immunohistochemistry.

### Immunohistochemistry:

Postmortem frontal cortex tissue of both humans and rhesus macaques was formalin-fixed and paraffin embedded for immunohistochemistry analyses. The dual staining immunohistochemistry protocol utilized in these studies has been previously described in Pitcher et al., 2013, Multispectral Imaging and Automated Laser Capture Microdissection of Human Cortical Neurons: A Quantitative Study of CXCR4 Expression. In Chemokines: Methods and Protocols. U. E. Cardona A, editor: Springer 31-48. Tissue was sequentially dual-stained with the neuronal marker MAP-2 (Chemicon, 1:250) and the protein of interest: FHC (Abcam, 1:50), pCXCR4-Ser339 (Abcam, 1:50) or CXCR4 (1:100). Following rehydration, endogenous peroxidase activity was quenched with hydrogen peroxidase and methanol for 30 min at room temperature, followed by antigen retrieval in a citrate buffer at 95° for 1 hour (DAKO). Tissue was blocked with avidinbiotin (Vector Labs) and blocking buffer before incubation with primary antibody overnight at 4° in a humidity chamber. Secondary antibody incubation (Jackson ImmunoResearch, 1:250) was performed for 2 hours at room temperature, then amplified with the avidin-biotin complex kit (Vector Labs). Visualization of MAP2 was performed with the hydrogen peroxidase conjugated NovaRed (Vector Labs), while the additional protein of interest (FHC, pCXCR4 or CXCR4) was visualized with the alkaline phosphatase conjugated VectorBlue (Vector Labs).

### Multispectral Imaging:

Imaging and analysis of dual stained human and rhesus macaque cortex may be outlined as follows. Briefly, bright field microscopy coupled with multispectral image analysis was performed to identify individual MAP2+ neurons within the cortex and measure the absolute optical density of the given antigen of interest—FHC, CXCR4 or pCXCR4-Ser339 (pCXCR4)—within each individual MAP2+ neuron. The absolute optical density is a semi-quantitative measure of protein expression on a cell-by-cell basis.

### Neuronal Cultures.

Rat cortical neurons were cultured in the presence of a glial feeder layer, using the bilaminar cell culture model. Due to the presence of glia this culture system largely preserves the in vivo mechanisms of neuronal growth and differentiation, but also allows for direct studies on neurons, which were separated from glia at the time of analysis. Selected experiments were performed using glia-free neuronal cultures, as noted, using previously described protocols (Sengupta et al., 2009, J. Neurosci. 29:2534-2544). Neurons were transfected at the time of plating by Nucleofection (Amaxa), or after 8 days in culture by lipofectamine (Invitrogen). All experiments were conducted at 21 DIV, unless otherwise noted. HDAC4 and control (scrambled) siRNA were obtained from Ambion (s170677 and 4390843), and FHC/scrambled shRNA from GenScript (custom order; shRNA vector also expressed GFP).

### Human Osteosarcoma (HOS) Cells:

CXCR4-expressing HOS cells were obtained through the AIDS Research and Reference Reagent Program, Division of AIDS, NIAID, NIH: HOS.CXCR-4 from Dr. Nathaniel Landau. Cells were grown in DMEM containing 10% FBS, 50 μg/mL gentamicin, and 1 g/mL puromycin. Experiments were conducted at ˜75% confluency, and cells were serum-starved for 24 hours before treatments. Transfections were performed by Nucleofection, and transfected cells were selected with 400 μg/mL G-418 to generate stable cell lines.

### Western Blot:

Total and nuclear extracts were obtained by standard protocols with minor modifications. Briefly, for total extracts, cells or tissues were lysed in lysis buffer containing 150 mM NaCl, 50 mM Tris, 0.5% Na deoxycholate, 0.1% SDS, 10 mM Na₄P₂O₇, 5 mM EDTA, 1% Triton-X, 1 mM DTT, and protease/phosphatase inhibitors. Equal amounts of protein (40-50 μg) were loaded into each lane for SDS-PAGE, and transferred to PVDF membranes for immunoblotting. For nuclear extracts, a hypotonic buffer solution, containing Tris-HCl (20 mM; pH=7.4), NaCl (10 mM) and MgCl₂ (3 mM), was used to isolate nuclei (0.05% NP40 was added toward the end of the incubation period). Nuclei were then lysed using a triple detergent buffer containing: 100 mM Tris, pH7.4; 2 mM Na3VO4; 100 mM NaCl; 1% Triton X-100; 1 mM EDTA; 10% glycerol; 1 mM EGTA; 0.1% SDS; 1 mM NaF; 0.5% deoxycholate; 20 nM Na₄P₂O₇. Protease and phosphatases inhibitors were added to both buffers. Histone H3 was used as nuclear marker; 20 ug of proteins were loaded into each lane.

Antibodies used: anti-FHC (H-53, Santa Cruz Biotechnology, 1:1,000), anti-pAkt (Ser473, Cell Signaling Technology, 1:2,000), anti-pCXCR4 (Ser339, Abcam, 1:1,000), anti-HDAC4 (A-4004, Epigentek, 1:1,000), anti-HDAC1 (10E2, Santa Cruz Biotechnology, 1:1,000), anti-β-actin (Sigma-Aldrich, 1:5,000) and anti-Histone H3 (9715, Cell Signaling, 1:1,000).

### Calcein Assay:

HOS cells transfected with either empty vector (EV), wild type FHC (WT-FHC) or FHC 222 mutant (Mut-FHC) were plated in 96-well plates at 20,000 cells per well. After 24 hours of serum starvation, vehicle or the iron chelator DFO (100 M) were added, respectively, to the untreated EV wells (Control) and to the positive control wells (DFO). Six hours later all cells were washed with PBS, and loaded with the iron-binding dye calcein (200 μg/mL) for 20 minutes. Cells were then washed again in PBS, and calcein fluorescence measured (excitation 485 nm, emission 535 nm), as a measure of the labile iron pool.

### Dendritic Spine Analysis

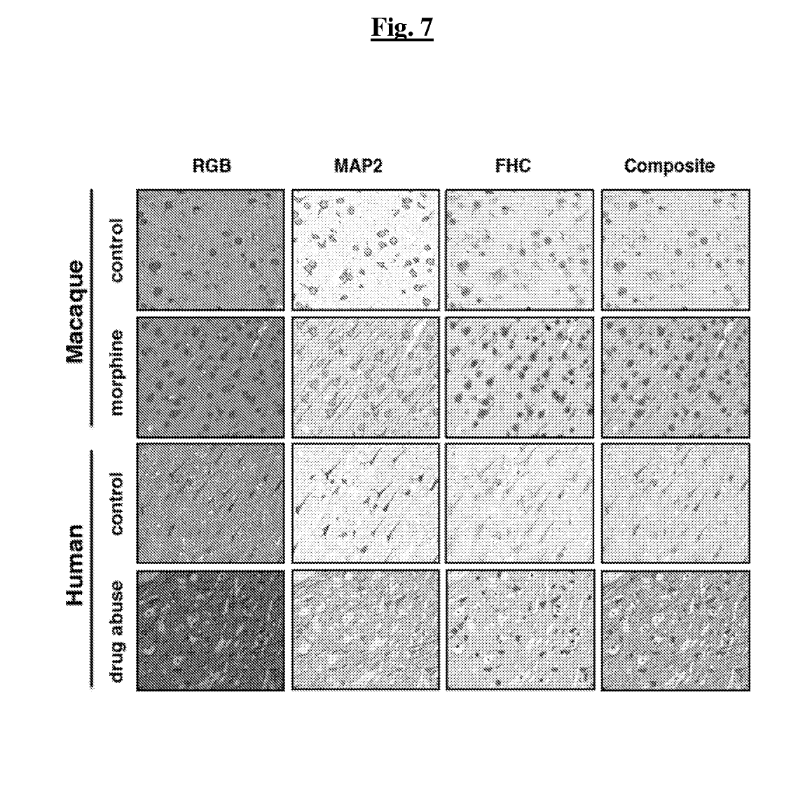

For in vitro experiments, neurons were cultured in the presence of glia for 21 days, then fixed and stained for MAP2 (Millipore, 1:1000; detected with Goat antirabbit Alexa 568, Invitrogen 1:250) and counterstained with phalloidin (Invitrogen, 25 μg/mL). For FHC shRNA experiments neurons were identified and analyzed using incorporated GFP expression. Images were acquired with a Zeiss LSM5 laser scanning confocal microscope, using a 63× objective. Dendritic spines were defined as phalloidin- or GFP-positive protrusions clearly connected to the dendrite by a thin shaft. For each neuron 3-4 segments totaling at least 200 μM in length were added to measure spine density, and each neuron was represented as a single data point. Each experiment included 3-4 coverslips from each of 2 or more separate preparations. For in vivo experiments, dendritic spines were visualized using a Golgi stain kit, following instructions from the manufacturer (FD NeuroTechnologies). Brains were rapidly removed, rinsed in H2O, and incubated in the Golgi stain for 2 weeks. The tissue was then cryoprotected in sucrose at 4° C. for 7 days, and frozen in isopentane. Brains were sectioned by cryostat at a thickness of 180 μm, and mounted on gelatin-coated microscope slides. Sections were allowed to dry overnight, and a signal intensification step performed before dehydrating, clearing in xylene, and coversliping with Permount (Fisher Scientific). Spine analysis was performed using Neurolucida software equipped with a 100× objective (FIG. 7A). Pyramidal neurons in layer 2-3 of prefrontal cortex were identified and reconstructed in their entirety, and total spine density for each neuron was reported as a single data point. 3 neurons were analyzed from each brain, and 4-5 brains used for each experimental group.

### Immunocytochemistry:

Neurons were fixed in 4% paraformaldehyde, and stained with MAP2 (Millipore, 1:1000) and HDAC4 (Epigentek, 1:150), detected with cy2- and cy3-conjugated secondary antibodies. Cell nuclei were visualized with Hoechst (Invitrogen, 1:2000), and images were acquired with a Zeiss LSM5 laser scanning confocal microscope (Imager.Z1m), mounting 10, 20, 40, and 63× objectives.

### Morphine Pellet Implantation:

Slow-release morphine or placebo pellets were implanted subcutaneously in 3-week-old rats, following an “escalating” dose protocol. Animals were anesthetized with a mixture of ketamine (80 mg/kg) and xylazine (10 mg/kg), and a small incision made through the skin to produce a small pocket under the skin. A single pellet of morphine (25 mg) or vehicle was placed inside, and the incision closed using 7 mm wound clips. 48 hours after this initial surgery, 2 additional pellets of morphine or vehicle were implanted in a similar manner. 48 hours after this second surgery, animals were sacrificed and brain tissue rapidly removed and processed.

### Intracerebroventricular Injections:

3-week-old rats received injections of AMD3100 (1 μg in 5 μl) or PBS into the left lateral ventricle. Animals were anesthetized with isofluorane, and stereotaxic coordinates zeroed to the interaural line. The following coordinates were used to locate the lateral ventricle: 5.5 mm anterior, 1.4 mm lateral, 6.4 mm dorsal. 5 μL of drug or PBS was mechanically injected over 10 minutes through a 33GA Hamilton needle. 6 hours after each injection, animals were sacrificed, and the brains rapidly removed and processed. Correct targeting of the lateral ventricle was verified by a similar injection of 2% Evans blue dye (FIG. 7B).

### Osmotic Pump Implantation:

Micro-osmotic pumps (Alzet) together with brain infusion kits (Alzet, Brain Infusion Kit 2) were implanted in 3-week-old rats to deliver AMD3100 (0.75 μg/hour) or PBS to the left lateral ventricle of the brain for a prolonged time period (4 days). The surgical procedure was similar to that of the intracerebroventricular injections, detailed elsewhere herein, although a cannula needle and osmotic pump replaced the Hamilton needle and syringe. The pump was placed in a subcutaneous pocket in the midscapular area, and a cannula was attached to the skull using a cyanoacrylate adhesive (Alzet). After 96 hours of continuous drug delivery (0.5 μL per hour), animals were sacrificed, and the brains rapidly removed and processed.

### Electrophysiology:

Experiments were conducted as previously described in Wang & Gao, 2010, J. Physiol. 588:2823-2838. For AMD3100 experiments, 6 hours following intracerebroventricular (ICV) infusion of AMD3100 (1 μg in 5 μl or PBS), animals were anesthetized with euthasol (0.2 ml kg⁻¹, I.P.) and perfused with ice-cold, oxygenated artificial cerebrospinal fluid (ACSF, in mM: 124 NaCl, 2.5 KCl, 1.25 NaH₂PO₄, 2 CaCl₂, 1 MgSO₄, 26 NaHCO₃, and 10 dextrose, pH 7.4). The brains were removed and horizontal sections through the prefrontal cortex were cut (300 μm) into an ice-cold bath of oxygenated ACSF using a vibrotome tissue slicer (Leica Microsystems, Buffalo Grove, Ill.).

Slices were transferred to a holding chamber, submerged in oxygenated ACSF at 35° C. for one hour and then remained at room temperature until used for recording. For CXCL12 experiments, prefrontal slices were generated as above, however the slices were transferred into a holding chamber containing vehicle or CXCL12 (20 nM) and incubated for 3 hours before recording began. Slices were then placed into a recording chamber mounted on an Olympus upright microscope (BX51, Center Valley, Pa.), where they were bathed in oxygenated ACSF at a flow rate of 2-3 ml/min. Neurons were visualized with infrared differential interference video microscopy. Somatic whole-cell voltage-clamp recordings were obtained from visually identified layer 2/3 pyramidal cells using patch electrodes with an open tip resistance of 5-7 MΩ and filled with a potassium gluconate internal solution (in mM: 120 potassium gluconate, 20 KCl, 4 ATPNa, 0.3 Na₂GTP, 5 Na-phosphocreatine, 0.1 EGTA, 10 Hepes, pH 7.3, 305 mosmol L⁻¹).

Whole cell current clamp was used to record action potentials in response to varying step currents from −300 pA to +400 pA with 50 pA increment. The recording was then switched into voltage clamp mode with membrane potentials held at −70 mV in the presence of picrotoxin (100 μM) to record spontaneous EPSCs (sEPSCs), or with both picrotoxin and tetrodotoxin (TTX, 1.0 μM).

### Example 1

### Alteration of Disease State by Expression of FHC Protein in Human Brain Cortical Neurons

In order to assess alterations in FHC and CXCR4 signaling within human HIV+ and drug-abusing individuals, postmortem samples of human frontal cortex were obtained from these patient populations. The human cohort was divided into four disease groups based on HIV status and illicit opiate drug use (DU): DU−/HIV− controls (N=14), DU+/HIV−(N=7), DU−/HIV+(N=19), DU+/HIV+(N=11) (see Tables 2-3).

As revealed by immunohistochemistry and multispectral image analysis, in which the optical density (OD) of FHC was quantified specifically within cells staining positive for the neuronal marker MAP2 (FIG. 7), the basal expression of FHC protein within human cortical neurons is normally low; within the control samples the absolute OD of the FHC:chromogen complex is mainly distributed just above the level of detection (FIG. 1A, left panel). In individuals with a history of drug abuse the mean FHC expression is significantly increased, and also shows increased variability. HIV+ individuals, both with and without a history of drug abuse, also show a significant elevation in mean neuronal FHC when compared to controls (FIG. 1A, right panel). Interestingly, the FHC expression within neurons is skewed toward high levels, particularly within the drug use only group, suggesting that the degree of FHC elevation within neurons is variable, and may depend on the underlying pathological cause (i.e. drug use or HIV). These data demonstrate an association of HIV with elevated neuronal FHC in the absence of drug use. Combined HIV infection and drug use similarly associates with a significant elevation of FHC protein, with comparable mean levels between the three disease groups (DU+, HIV+, DU+/HIV+).

### Example 2

### Reduction of CXCR4 Phosphorylation in Human Brain Cortical Neurons by HIV and Drug Abuse

Phosphorylation of the human chemokine receptor CXCR4 at Ser-339 in the C-terminus results from activation by the endogenous CXCR4 ligand CXCL12, and its presence indicates proper functioning of the CXCL12/CXCR4 signaling axis. In order to assess CXCR4 function in relation to the FHC findings, adjacent tissue sections were stained using an antibody that targets CXCR4 phosphorylated specifically at Ser-339 (pCXCR4), and pCXCR4 levels within MAP2+ neurons were compared across disease groups (FIG. 1B). A significant reduction in mean neuronal pCXCR4 levels was observed in individuals with either a history of drug use or HIV, as well as individuals with a history of both drug use and HIV.

Certain disease states have been shown to alter neuronal CXCR4 expression; therefore low levels of pCXCR4 could be a consequence of low overall CXCR4 levels rather than an inhibition of receptor activation. Total neuronal CXCR4 levels were therefore also measured, revealing no reduction in total CXCR4 within any disease group (FIG. 1C). These data indicate that the reduction of pCXCR4 seen in patients with HIV or a history of drug use is reflective of an inhibition of receptor activity, rather than a regulation of receptor expression. Interestingly, however, the HIV+ and DU+/HIV+ disease groups both displayed significantly higher total CXCR4 levels, and suggest that the relative activation of CXCR4 in the HIV+ and DU+/HIV+ groups is disproportionally lower than in the DU+ group alone. These findings suggest that an additive or synergistic disruption of neuronal CXCL12/CXCR4 signaling may exist between HIV and opiate use, despite the roughly comparable levels of pCXCR4 between the DU+, HIV+ and DU+/HIV+ groups.

### Example 3

### Negative Correlation Between FHC Negatively and pCXCR4 In Vivo

The findings linking elevated neuronal FHC to both drug use and HIV infection, independently, suggest that both MOR-dependent and MOR-independent pathways regulate FHC during pathological conditions. In each of these states, however, an inverse relationship between the OD of FHC and the OD of pCXCR4 within individual patients can be expected, as a consequence of FHC's ability to regulate CXCR4 signaling.

In an additional study of a subset of human patients, the neuronal expression of pCXCR4 and FHC within individual patients was plotted, revealing a significant inverse relationship between FHC and pCXCR4 levels (N=16; Pearson correlation=−0.724, p<0.01) (FIG. 1D). This result is consistent with an important role of FHC in CXCR4 inhibition in vivo.

Collectively, these data show that certain disease states, such as drug use (including opiate use) or CNS infection (HIV), abnormally elevate neuronal FHC, resulting in disrupted function of the CXCL12/CXCR4 signaling axis.

### Example 4

### Correlation of FHC with demographic and clinical data

Expression of FHC protein can increase with age; therefore age could be a potential confounding variable affecting these results. The age of the patient cohort ranges from 30 to 73 years old (FIG. 1E), with an average of 47.5. Correlations between age of death and average neuronal FHC expression levels for each patient were investigated among the four groups, and no significant correlation was found within any group (FIG. 1E), indicating that the elevated FHC expression is not due to advanced age. Although this finding cannot necessarily be generalized across pediatric or geriatric patient populations, it suggests that neurocognitive impairment seen in the largest HIV+ age group within the USA, 45-49 years old, is not due to age-related elevation of FHC within the CNS.

In order to determine clinical correlations between neuronal FHC and other important HIV biomarkers more broadly, a correlational matrix was generated for the patient cohort, including each patient's neuronal FHC OD, viral load within the cerebrospinal fluid (CSF) and peripheral blood (PB), CD4 and CD8 cell counts, and Memorial Sloan Kettering (MSK) score, a clinical measure of cognitive impairment (Table 1). The data show that cognitive impairment correlated significantly with neuronal FHC, but not with any other measurements, and suggest that FHC alterations may be a sensitive indicator of HAND neuropathogenesis.

### Example 5

### Independent Regulation of Neuronal FHC within Non-Human Primates by Morphine and SIV

HAND is a highly complex pathogenic process, further complicated in drug-using patient populations by widespread polydrug abuse. Several factors that could influence the regulation of neuronal FHC, such as opiate dosage, frequency of use, and time between the last administration and death, may additionally increase variability within our human cohort.

In order to control for these inherent variables, the previously described methods were applied to a nonhuman primate (NHP) model of SIV/opiate-induced neuropathology. Four groups of Indian Rhesus macaques were studied, respectively treated with morphine, infected with SIV, given both morphine and SIV, or delivered a control saline only (Table 4). Treatments began with intravenous SIV infection, using the SIV strain SIVMac251 (or vehicle), which was followed by a three-month regimen of morphine. The animals were then sacrificed, and tissue sections processed and stained using identical protocols and reagents as the human tissue.

As with the human samples, neuronal FHC protein expression within the macaques was measured as the OD of FHC quantified specifically within MAP2+ neurons. Consistent with the human data, neuronal FHC was significantly elevated in animals that received morphine (FIG. 2A). The variation, however, within the morphine treated macaques was reduced as compared to that of the human DU group, likely a reflection of both greater homogeneity among the animal population and a strictly controlled mono-drug treatment. Importantly, the significant elevation of neuronal FHC following administration of morphine alone, without other drugs of abuse, argues strongly for a mechanistic role of opioid receptors in neuronal FHC regulation. SIV-infected animals also showed a significant elevation of FHC within cortical neurons; however, in this group, the variability of FHC within neurons was greater than in the group treated with morphine alone. Animals that were both infected with SIV and treated with morphine also showed a trend for elevated neuronal FHC, comparable to the morphine and SIV groups. Overall, the macaque model was consistent with respect to the human population and supports the hypothesis that morphine and HIV/SIV elevate neuronal FHC in vivo.

### Example 6

### Correlation of FHC with Reduced pCXCR4 within the Non-Human Primate Model

Also consistent with the human data, macaques treated with morphine display a significant reduction in pCXCR4 levels, within a comparatively small range (FIG. 2B). SIV infected animals additionally show a significant decrease in pCXCR4, however, as with FHC, the range of pCXCR4 expression is greater than that of morphine-treated animals. Neuronal pCXCR4 also trended to decrease in animals that were both infected with SIV and treated with morphine.

The overall expression of neuronal CXCR4 was measured to confirm that the decreased expression of pCXCR4 is not due to a decrease in overall neuronal CXCR4 within these animals. Levels of total neuronal CXCR4 were unchanged in the morphine treated group, but elevated in SIV infected animals (FIG. 2C); reduced levels of pCXCR4 cannot thus be explained by a reduction in overall receptor levels. Both human HIV infection and macaque SIV infection resulted in a significant increase in neuronal CXCR4, suggesting that SIV, like HIV, causes a dysregulation of neuronal CXCR4 expression independent from morphine treatment. As in the human groups, comparing the mean neuronal FHC expression with mean neuronal pCXCR4 expression reveals a significant negative correlation (N=15 animals; Pearson correlation=−0.547, p=0.035) (FIG. 2D), further supporting the notion that pathologic alterations in pCXCR4 are associated with changes in FHC.

### Example 7

### Correlations of Neuronal FHC with Disease Status

The 4 animals only infected with SIV wee easily stratified by FHC expression such that two animals showed substantially higher FHC levels than the other two, suggesting the presence of two distinct populations within this SIV treated group. The two animals with higher FHC levels showed lower pCXCR4 levels, supporting a correlation between neuronal FHC and pCXCR4 levels, even within a single group (FIG. 2E). After multispectral image analysis the corresponding clinical data was unblinded, revealing that the higher FHC expressers retained an elevated viral load following inoculation, while the lower FHC expressers did not. Additionally, at the time of sacrifice histopathological evidence of HIV encephalitis (HIVE) was not yet present in the SIV animals, suggesting that FHC elevation may precede pathological changes associated with HIVE. Together these findings reveal a consistent association of neuronal FHC induction and concomitant CXCR4 inhibition with HIV disease progression, which may play an important role in HIV neuropathogenesis.

### Example 8

### Role of Iron Binding Activity in FHC Regulation of CXCR4

The ability of FHC to regulate CXCR4 signaling, an activity that has been observed in multiple cell types including neurons, may occur independently from the protein's well-characterized iron sequestering functions. It was investigated whether this effect of FHC was a consequence of its iron binding activity, or a truly novel function of the protein. This was addressed by comparing the CXCR4-regulatory effects of mutant FHC 222, a mutated form of FHC unable to bind or oxidize iron, to those of wild-type FHC.

Human osteosarcoma cells expressing high levels of CXCR4 (HOS-X4) were transfected with either of these FHC plasmids and selected to generate stable cell lines. First, in order to confirm the differential effects of these proteins on iron sequestration, free intracellular iron levels were compared using the fluorescent probe calcein. Calcein is delivered as a calcein-AM ester, which passes across the plasma membrane into cells and is cleaved by cytosolic esterases to produce membrane-impermeable calcein. The fluorescence of this trapped calcein is quenched following chelation with labile iron, and therefore the degree of quenching is a commonly used estimation of free intracellular iron levels.

Cells overexpressing wild-type FHC displayed greater calcein fluorescence than control cells, similar to cells treated with the iron chelator DFO. Calcein fluorescence was not, however, altered in cells expressing the mutant FHC 222, consistent with the inability of this mutant to bind iron (FIG. 3A). The effects of the wild-type and mutant FHC protein on CXCR4 signaling were then compared by treating each cell line with CXCL12 and measuring the activation of downstream signaling pathways. As summarized in the two graphs of FIG. 3B, phosphorylation of CXCR4 (left) and activation of its downstream target Akt (right) in response to CXCL12 were reduced both in cells expressing wild-type FHC and in cells expressing mutant FHC 222, suggesting that CXCR4 regulation is an independent function of FHC, distinct from the protein's primary iron-binding activity.

### Example 9

### Regulation of Dendritic Spine Density In Vitro and In Vivo by CXCL12/CXCR4

CXCL12/CXCR4 axis protects neurons from various insults involved in HIV-induced neuronal damage and ensuing neurological disorders, such as exposure to the HIV-envelope protein gp120 or excessive glutamate stimulation. A particularly important component of HAND is the reduced complexity of the dendritic arbor and synaptic density, including a loss of dendritic spines, which occurs early in the disease and is thought to contribute directly to cognitive loss. Additionally, CXCL12 regulates p21-activated kinase (PAK) in T-cells, a kinase which contributes to morphological changes in these cells, and which promotes dendritic spine formation in neurons.

Studies were performed to evaluate the possible direct role of CXCL12 in the regulation of dendritic spine density, both in vitro and in vivo. Rat cortical neurons in culture were first treated with CXCL12 (in the presence of glia), and a moderate but significant increase in spine density was observed 3 hours after a single dose (FIG. 4A). This effect of CXCL12 on spine density was confirmed at multiple time points in a pure neuronal culture lacking a glial feeder layer, indicating a direct effect of CXCL12 on neurons (FIG. 4B). Additionally, this effect of CXCL12 was blocked by the specific CXCR4 antagonist AMD3100 and by pertussis toxin, an inhibitor of Gαi signaling (FIG. 8), supporting the involvement of CXCR4 and its G-protein dependent signaling.

Histone deacetylases (HDACs) are thought to play an important role in mediating the neuroprotective functions of CXCR4 activation. HDAC4 play a role in the regulation of dendritic spines by CXCL12/CXCR4, as evidenced by the fact that this HDAC promotes neuronal survival and synaptic plasticity, and that the transcription factor myocyte enhancer factor 2 (MEF2), the major nuclear target of HDAC4, is directly involved in structural synapse regulation. Like the other Class II HDACs, HDAC4 activity is regulated by nuclear import/export.

It is thus possible that an increase in nuclear HDAC4 levels mediates the effects of CXCL12 on spine density. Consistent with this hypothesis, neurons cultured as a pure population and treated with CXCL12 exhibited increased nuclear HDAC4 levels, as shown by immunocytochemistry and western blot studies (FIG. 4C). In order to assess the involvement of HDAC4 in CXCL12-induced spine alterations, HDAC4 levels were reduced by transfecting neurons with HDAC4-specific siRNA (FIG. 4D). The resulting HDAC4 deficiency completely prevented the effects of CXCL12 on spine density, indicating a crucial role of this HDAC in CXCL12-induced spine regulation.

In order to confirm the spine regulating effect of CXCL12 in vivo, in the context of opiate abuse and decreased CXCR4 signaling, the CXCR4 antagonist AMD3100 was administered to rats through intracerebroventricular (ICV) injections. Six hours after a single 1 μg injection of this drug, dendritic spine densities were analyzed among layer II/III pyramidal neurons of the prefrontal cortex, by reconstructing individual neurons in their entirety. This single dose of AMD3100 significantly decreased spine densities, indicating that the basal level of CXCR4 signaling contributes to the regulation of spine density in vivo (FIG. 4E). In order to investigate the effects of more prolonged CXCR4 inhibition, as would occur with long-term opiate abuse, osmotic pumps were implanted to deliver constant levels of AMD3100 into the left lateral ventricle of the brain for 4 days (FIG. 4F). Following this type of treatment AMD3100 was similarly able to decrease dendritic spine densities. Together these data identify the endogenous CXCL12/CXCR4 axis as a novel regulator of dendritic spines, and suggest a specific neuropathological outcome of CXCR4 dysregulation highly relevant to opiate abuse and neuroAIDS.

To determine the functional consequences of spine density alterations, whole cell patch clamp electrophysiology was used to record from layer 2/3 pyramidal neurons in the prefrontal cortex. First in current clamp mode, action potentials were recorded in response to varying step currents in slices from animals that had received ICV injections of AMD3100 or vehicle, or from slices that had been exposed ex vivo to CXCL12 or vehicle. For both drug conditions, there was no drug effect on spike number generation (AMD3100, p=0.57; CXCL12, p=0.71). Furthermore, there was no drug effect on resting membrane potential, AP threshold, AP peak amplitude, AP 1/2 width, 20-80% rise time, and the after hyperpolarization (Table 5). Thus, drug exposure did not significantly alter cell health or basic functional properties. Spontaneous and miniature excitatory postsynaptic currents (sEPSCs and mEPSCs) were recorded while holding the cell at −70 mV (FIG. 5). AMD3100 had no effect on either the frequency or amplitude of the sEPSCs that are presumably resulted from action potentials of presynaptic neurons as well as spontaneous release of neurotransmitter at synapses (AMD3100, sEPSCs: frequency, Hz, vehicle 4.22±0.70, AMD3100 3.84±0.65, p=0.69; amplitude, pA, vehicle 18.87±1.41, AMD3100 16.79±0.80, p=0.10; n=6). However, AMD3100 exposure significantly reduced the frequency of mEPSCs and induced a trend towards reduced mEPSC amplitude (frequency, Hz, vehicle 3.47±0.41, AMD3100 2.27±0.33, p<0.04; amplitude, pA, vehicle 18.3±0.52, AMD3100 16.55±0.55, p=0.068; n=10 and 9 respectively). mEPSCs are the result of individual synaptic events and thus their frequency may be an indication of the number of functional synapses. Therefore, these data are consistent with a reduced number of synapses that would be expected with the reduced dendritic spine density following the AMD3100 treatment. Further, the trend toward a decrease in mEPSC amplitude may indicate changes to post-synaptic receptors. Similarly, sEPSCs and mEPSCs were recorded following exposure of slices to CXCL12. There was no difference in the frequency of sEPSCs or mEPSCs (sEPSCs, frequency, Hz, vehicle 2.07±0.36, CXCL12 1.85±0.41, p=0.64, n=6 and 7, respectively; mEPSCs, frequency, Hz, vehicle 1.59±0.24, CXCL12 1.43±0.25, p=0.66, n=8). This lack of effect may be the result of an altered time course of CXCL12 on slices versus primary culture. Another factor is the potential effect of CXCL12 on GABAergic neurotransmission, which may indirectly affect dendritic spines (56); GABA receptors are inhibited by picrotoxin present in the recording solution but not in the culture studies.

In contrast, CXCL12 significantly increased sEPSC amplitude and there was a trend towards an increase for mEPSC amplitude (sEPSC, pA, vehicle 23.34±1.07, CXCL12 25.32±0.43, p=0.027; n=6 and 7 respectively; mEPSC, vehicle 20.873±1.21, CXCL12 25.25±1.02, p=0.051, n=8). Together these data suggest the CXCL12/CXCR4 axis regulates functional connectivity of layer 2/3 pyramidal neurons and that stimulation of this receptor by the endogenous ligand may alter both spine density and synaptic strength.

### Example 10

### Decrease of Dendritic Spine Density Through Effects on Neuronal FHC Using Morphine

Opiates such as morphine reduce dendritic spine density and synaptic plasticity, which may contribute to cognitive dysfunction among vulnerable populations such as HIV+ opiate abusers. Furthermore, the findings that constitutive CXCR4 signaling is important in regulating spine number and neuronal activity suggest a specific FHC-mediated pathway by which opiates alter neuronal function.

The role of FHC in opiate-induced spine loss was investigated, first by confirming this effect both in vitro and in vivo. Morphine treatment significantly increases FHC levels in cultured rat cortical neurons (FIG. 6A). This effect was associated with a significant decrease in the number of dendritic spines (FIG. 6B), in line with reports showing collapse of spine and reduced mEPSC in neurons exposed to morphine. A similar effect in vivo was observed following a prolonged morphine treatment; rats were implanted with slow-release morphine pellets that delivered morphine continuously for 4 days, which was shown to both increase levels of FHC in the brain lysates (FIG. 6C), and significantly decrease dendritic spine densities in these same animals (FIG. 6D). In order to evaluate a direct effect of FHC on spine density, neurons were then transfected to over-express FHC (FIG. 6E). These neurons expressing high levels of FHC contained fewer dendritic spines, indicative of a pathological role of FHC in neuronal function and survival. Additionally, the role of FHC in morphine-induced spine loss was explored through the use of FHC-specific shRNA. The ability of morphine to regulate spines was blocked in FHC-deficient neurons (FIG. 6F), suggesting an important role of FHC in neuronal dysfunction resulting from opiate use.

### Example 11

### Induction of Interleukin-1 Beta by HIV-1 Viral Proteins Leads to Increased Levels of Neuronal Ferritin Heavy Chain and Synaptic Injury

### Materials and Methods

### Materials:

Cell culture media were purchased from Invitrogen. Morphine sulfate was obtained from Sigma-Aldrich. HIV-1_{IIIB} gp120 was acquired from Immunodiagnostics. Recombinant TNF-α, IL-1β, monoclonal anti-rat IL-1β/IL-1F2 antibody, monoclonal anti-rat TNF-α antibody, and recombinant rat IL-1ra/IL-1F3 were purchased from R&D Systems. The following reagent was obtained through the NIH AIDS Research and Reference Reagent Program, Division of AIDS, NIAID, NIH: HIV-1BaL gp120.

### Animals:

Animals, except Sprague-Dawley that are from Taconics Farm, were purchased from Harlan Laboratories (Indianapolis, Ind.). E17 Holtzman pregnant rats (or their P4 litters) were used for neuronal and glial cultures, respectively. HIV-Tg rats, Hsd:HIV-1(F344), and F344/NHsd age-matched controls were used to determine the in vivo effect of viral proteins expression on dendritic branching and spines. The HIV-Tg rats express 7 of the 9 HIV-1 proteins, including the envelope protein gp120 (Reid, et al., 2001, Proc Natl Acad Sci USA, 98(16):9271-6). Specifically, a 3-kbp SphI-MscI fragment encompassing the 3′ region of gag and the 5′ region of pol was removed from pNL4-3, an infectious proviral plasmid, to make the noninfectious HIV-1gag-pol clone pEVd1443 (Masliah, et al., 1997, Ann. Neurol. 42(6):963-72). Due to immunodeficiency, these animals were housed in isolation in the barrier facility. HIV-Tg rats develop neuropathological and inflammatory features similar to those observed in HAND, including elevated levels of pro-inflammatory cytokines and behavioral problems indicative of cognitive deficits (Vigorito, et al., 2007, J Neuroimmune Pharmacol. 2(4):319-28; Lashomb, et al., 2009, J. Neurovirol. 15(1):14-24.).

### Cannula Implant and Gp120 Infusions.

Rats (200-400 g, male Sprague-Dawley, Taconic Farms) were stereotaxically implanted, under isoflurane anesthesia, with stainless-steel cannulas placed 0.96 mm posterior and 3.18 mm lateral to bregma, 3.47 mm below the surface of the cranium at an angle of 22°. Four stainless-steel screws (#0-80) were placed around the cannula and acrylic dental cement was used to anchor the cannula. Animals were allowed seven days to recover before receiving a bilateral infusion of 1 μl of gp120_{IIIB} (50 ng/μl) once daily for seven days via a 10 microliter Hamilton Syringe, needle diameter 0.48 mm; infusion rate=0.374 microliters/minute. Animals were food restricted and began behavioral testing 21 days following the final infusion.

### Neuronal Cultures.

Rat cortical neurons were cultured in serum-free media in the presence of a glial feeder layer (Shimizu, et al., 2011, J. Vis. Exp. 57). This culture system largely preserves the in vivo environment, due to the presence of glia; however, it still enables direct investigation of neurons that can be separated by the glia before experiments. Selected studies were also performed in neurons grown in the absence of glia for the entire culture period, as indicated (Sengupta, et al., 2009, J. Neurosci. 29(8):2534-44). All experiments were conducted between 8-13 days in vitro (DIV).

### Western Blots:

Total protein extracts were obtained using standard protocols. Briefly, cells or tissue were lysed in lysis buffer containing 150 mM NaCl, 50 mM Tris, 0.5% Na deoxycholate, 0.1% SDS, 10 mM Na₄P207, 5 mM EDTA, 1% Triton-X, 1 mM DTT, and protease/phosphatase inhibitors. Equal amounts of protein (40-50 g) were loaded into each lane for SDS-PAGE and transferred to PVDF membranes for immunoblotting. The following antibodies were used: anti-FHC (3998, Cell Signaling Technology, 1:1,000) and anti-β-actin (A2066, Sigma-Aldrich, 1:5,000).

### ELISA:

TNF-α and IL-1β secreted protein levels were determined from co-culture supernatants using commercially available rat TNF-α and IL-1β kits (Quantikine, R&D Systems). The assays were performed following the manufacturer's instructions. Absorbance was read at 450 nm using Wallac Victor2 1420 Multilabel Counter (PerkinElmer). The concentration of secreted TNF-α and IL-1β are expressed as picograms per milliliter (pg/mL); values were normalized to total protein content for each well.

### Behavioral Testing:

Animals were tested in an attention set-shifting task (Birrell & Brown, 2000, J. Neurosci. 20(11); 4320-4324). On day 1 of testing, food restricted rats are habituated to the testing chamber for 20 minutes. The testing chamber (17″×22″×10″ H) is divided lengthwise by a plexiglass wall with a guillotine-style door creating a waiting area (one-third of the chamber), and a testing area (remaining two-thirds of the chamber). The far wall of the testing area (furthest from the waiting area) is bisected by a plexiglass divider that extended 9.25 inches; thus creating two distinct yet easily accessible sides of the testing area. The following day, rats are trained to dig in terra cotta pots to obtain a food reward (Frosted Cheerio). The food reward is progressively buried deeper in the pots, filled with corn cob bedding, until the rat reliably digs to retrieve the food reward. On day 3, rats undergo exemplar training, discriminating between sensory cues to determine which pot contains the food reward. Three pairs of pots that differ in only 1 dimension (sensory modality) are used during exemplar training: 2 pots with differing scents (almond vs snow), 2 pots with differing digging media (black string vs white string), and 2 pots with different textures on the exterior of the pots (suede vs cotton). The scent-paired pots are placed in the testing area (one on each side of the divider) and one of those pots is baited with food reward. At trial onset the guillotine door is raised allowing the rat to leave the waiting area, explore the testing area, and dig in one of the pots. If the rat begins digging in the incorrect pot, the pots are removed from the testing area, the rat is placed back into the waiting area, and the trial is recorded as incorrect. A dig is defined as vigorous displacement of the digging medium. If the rat digs in the correct pot, it is permitted to eat the food reward before being placed back in the waiting area. The pots are removed, the appropriate pot is re-baited with a food reward, and the pots are placed back in the testing area. The side of the baited pot is selected on a pseudo-random basis. An animal reaches criterion after digging for reward in the correct pot on six consecutive trials. The same procedure is repeated with the other two sets of pots. When a rat is exposed to a pair of pots for the first time, there are four discovery trials that allow the animal to explore both pots, dig without consequence, and do not count as choices.

Day four is the testing day, which consists of eight stages. Stage one begins with a simple discrimination in which the pair of pots differs in only 1 dimension. After reaching criterion, stage two is a complex discrimination where the pots differ in two dimensions (scent and digging media), but only one is relevant for reward discovery. Stage three is a reversal trial where the previously unrewarded cue in the same dimension that was used in stage two now becomes the rewarded cue. Stage four is an intradimensional shift where a new set of pots with entirely new scents, media, and textures are used, but the rewarded pot will remain in the same dimension as the previous stages. Stage five is another reversal trial conducted in the same manner as stage three's reversal trial. Following this is an extra-dimensional shift in stage six. During this stage, the previously rewarded dimension now becomes irrelevant while one of the cues in the previously irrelevant dimension becomes rewarded (e.g., if scent was the rewarded dimension for the first five stages, the digging media becomes the rewarded dimension). Stage seven is another reversal trial. Overall, the attention set shifting task requires recognition of rule changes and cognitive flexibility. It is analogous to the Wisconsin Card Sort used in human testing.

### Dendritic Spine Analysis:

Dendrites were visualized using a Golgi stain kit, according to the manufacturer's instructions (FD NeuroTechnologies). Briefly, brains were rapidly removed, rinsed in H₂O, and incubated in Golgi stain for 2 weeks. Tissue was then cryoprotected in sucrose at 4° C. for 7 days and frozen in isopentane. Brains were sectioned by cryostat at a thickness of 180 m and mounted on gelatin-coated microscope slides. Sections were allowed to dry overnight, and a signal intensification step was performed before dehydrating, clearing in xylene, and coverslipping with Permount (Fisher Scientific). Pyramidal neurons in layer II/III prefrontal cortex were identified and reconstructed in their entirety using a Zeiss Axio Imager M.2 microscope mounting a 100× oil immersion objective and equipped with Neurolucida software. Total spine density for each neuron was recorded and reported as a single data point. Number, length, and branching points of basal/apical dendrites were also scored. Four brains per experimental group were used and four whole neurons per brain were analyzed.

### Statistics:

Data are reported as mean±SEM. Statistical significance was determined by Students t-test or one-way ANOVA followed by Newman Keuls post-test. A p<0.05 was considered statistically significant. P values and n are reported in figure legends.

Dendritic Spine Density and Branching are Reduced in the Prefrontal Cortex of Rat Models of HAND and Correlate with FHC Changes and Cognitive Impairment

Synaptic injury, including reduction in dendritic arbor complexity and spine density, is a hallmark of HAND, being widely distributed throughout the HIV brain. However, synaptic injury appears predominant in some regions such as striatum, hippocampus, and prefrontal cortex (PFC). The development of small rodent animal models of HIV infection, including the HIV-1 Tg rat, has allowed the study of individual viral proteins, as well as the whole virus, on CNS complications of HIV. The non-infectious HIV-1 Tg rat expresses seven of the nine HIV-1 genes, both peripherally and centrally. Additionally, whole brain lysates from these animals show elevated levels of TNF-α and IL-1β protein and mRNA. gp160, the precursor for gp120 and gp41, is n found in the frontal cortex of these transgenic rats, which also show behavioral abnormalities consistent with neurocognitive deficit.

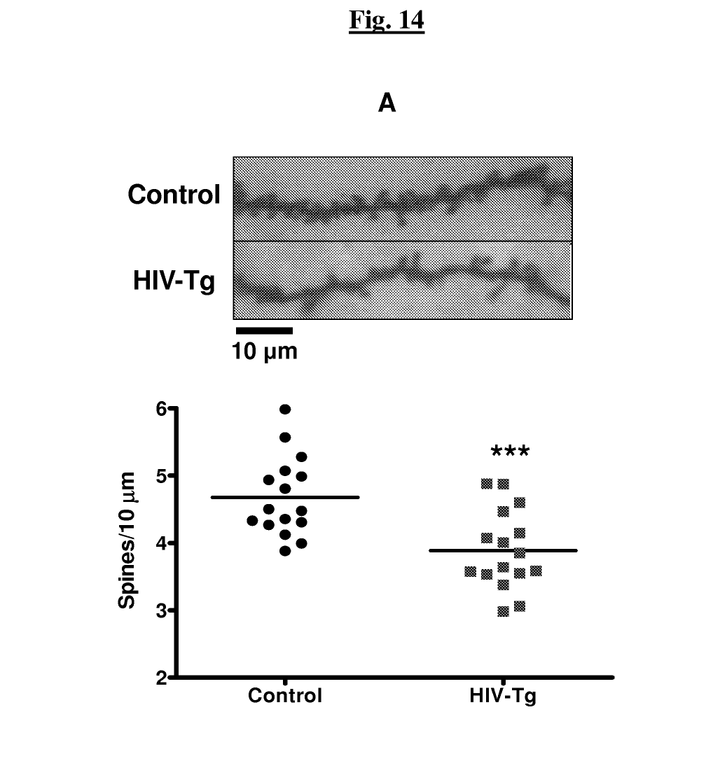

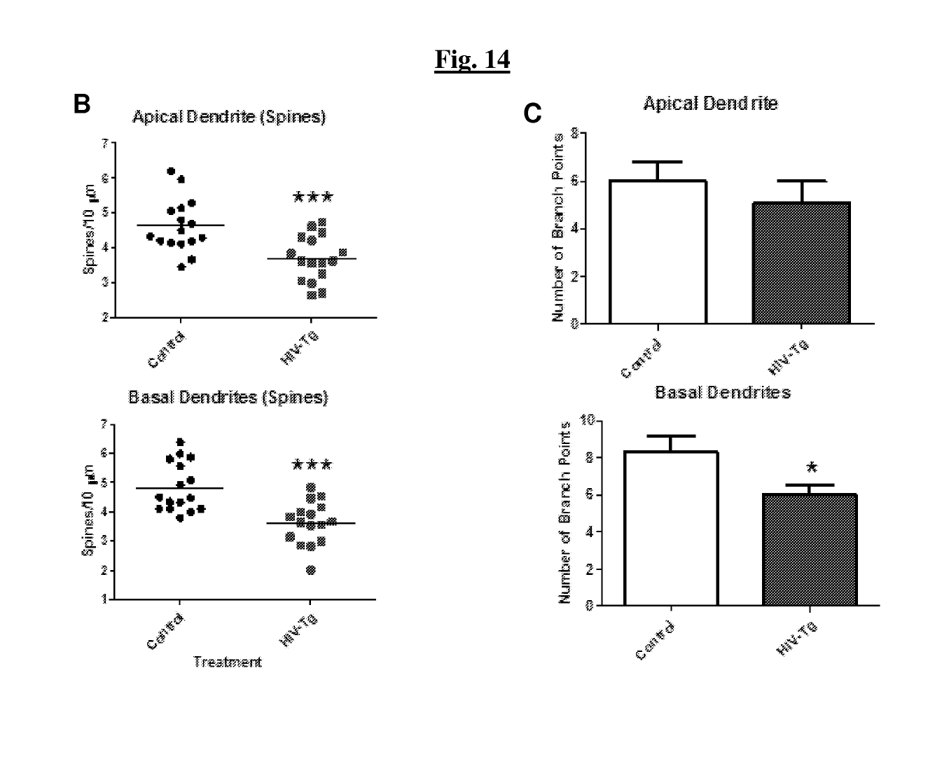

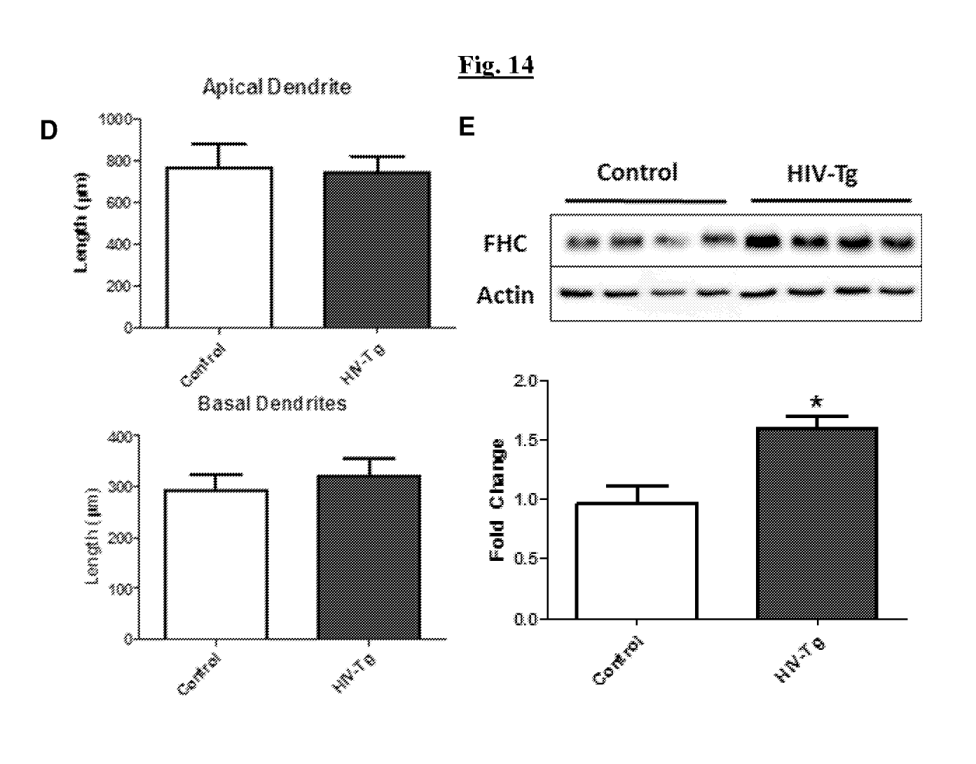

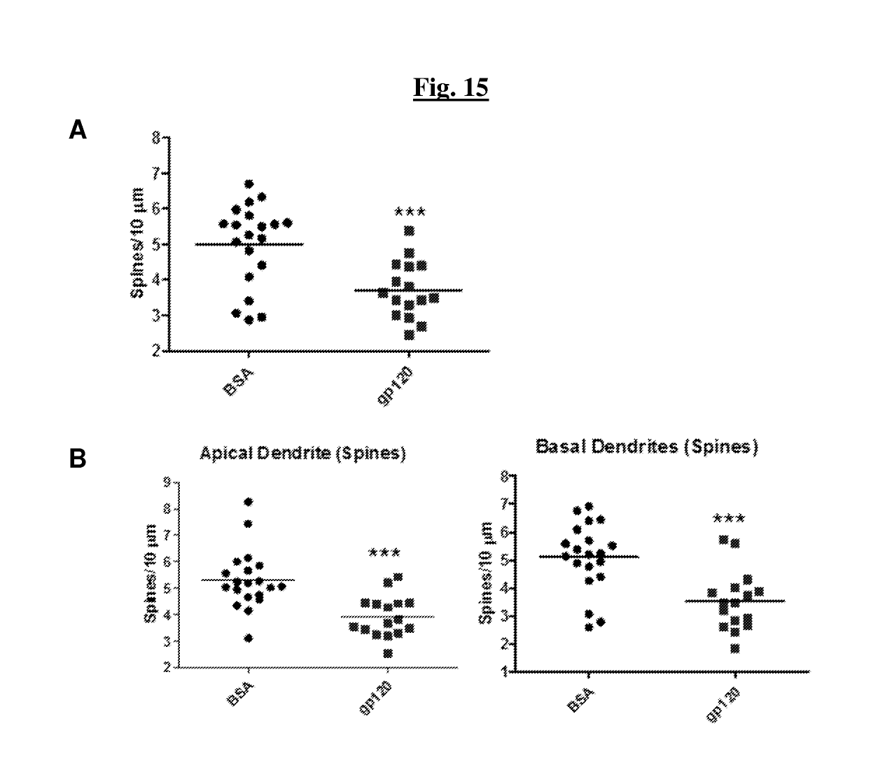

The possibility that dendritic branching and spine numbers are decreased in the PFC of HIV-1 Tg rats, as observed in post mortem human tissue, was investigated. Dendrites of both HIV-1 Tg rats and age/sex-matched controls were analyzed among layer II/III pyramidal neurons of the rat medial PFC, by reconstructing neurons in their entirety. Compared to controls, dendritic spine density was significantly lower in HIV-1 Tg rats (FIG. 14A), which indicates that the presence of viral proteins and/or secreted mediators contribute to decreased dendritic spine numbers. Spine density was reduced to a similar extent in apical and basal dendrites of HIV-1 Tg rat PFC neurons (FIG. 14B), and no differences in branch lengths were noted between wild type and Tg rats (FIG. 14D). However, the overall number of branches in the transgenic neurons was lower than in control neurons (FIG. 14C).

The levels of FHC in the brain of these animals were also examined. Levels of FHC were increased in HIV-1 Tg rat brains compared to controls. These findings suggest that, similar to human subjects, FHC is involved in the regulation of dendritic spine density in the cortex of HIV-1 Tg rats.

In order to narrow down the contribution of different viral proteins to the dendritic alterations observed, spine density, dendritic length, and branching in rats treated with HIV-1 gp120 were also examined. These animals were also examined for performance in a flexible attention task, the attention set-shifting, before processing for Golgi staining. Ten animals (n=5 SD and n=5 F344) were tested in the task after exposure to gp120_{IIIB} and seven animals (n=6 SD and n=1 F344) were tested after similar treatment with BSA (Table 5).

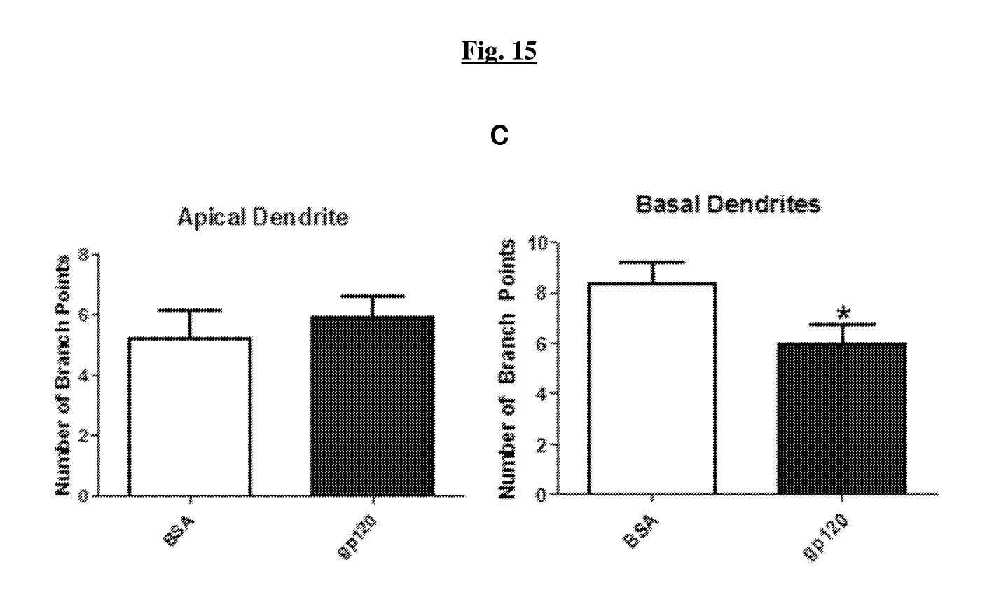

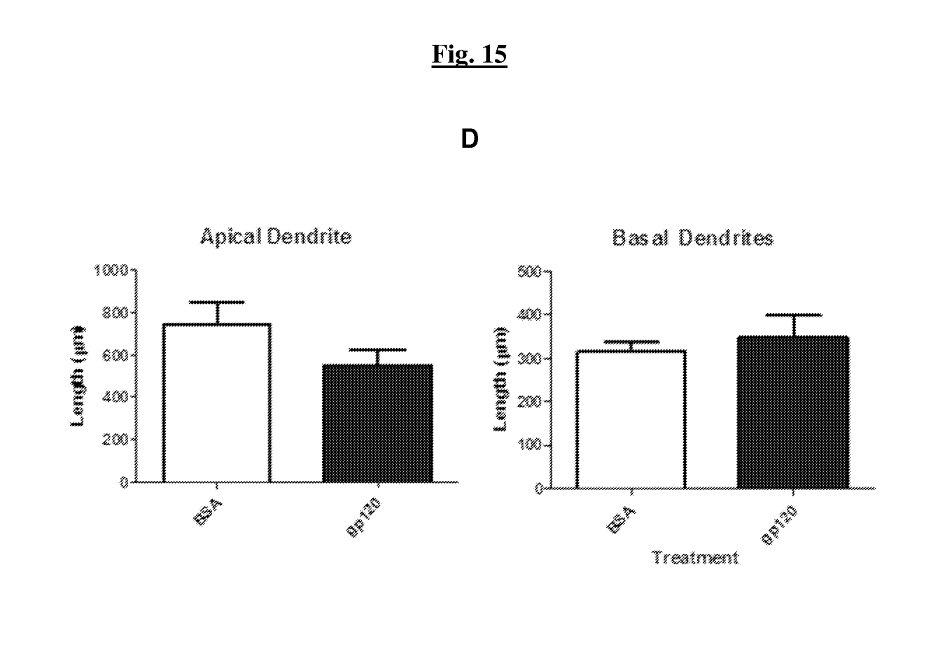

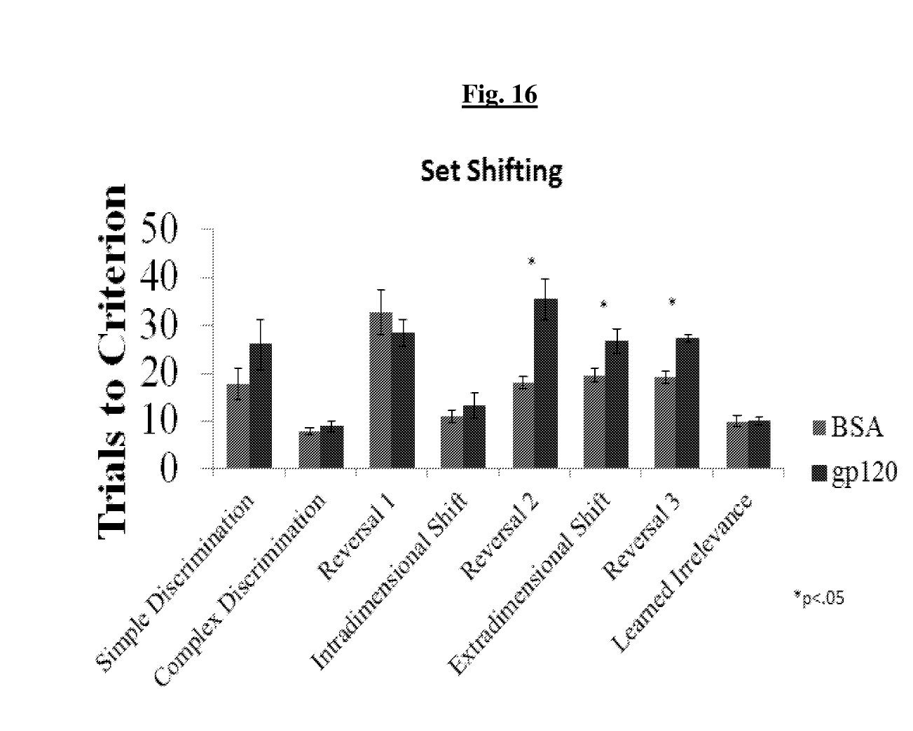

Three of the F344 rats that received gp 120 treatment were unable to reach criterion to complete the attention set-shifting task. One additional Fischer rat was unable to form sets. As a result, data from the Fischer 344 animals were not included in the statistical analyses. The remaining gp120 treated SD rats exhibited impaired attention set-shifting performance compared to BSA treated controls. More specifically, these animals were impaired in the reversal learning component of the task; Reversal 2 t(4.620)=4.035, p=0.012, a=0.05, Reversal 3 t(9)=5.767, p<0.001, a=0.005. Deficits in reversal learning occur following damage to the orbitofrontal cortex. Additionally, rats treated with gp120 were impaired in extra-dimensional shifts t(9)=4.392, p=0.026, α=0.05, a component of the task dependent on intact function of the medial prefrontal cortex. The performance of the gp120 treated rats in a flexible attention task correlated with the changes in the spines. Echoing the results in the HIV-Tg rats, the studies showed reduction of spine density in layer II/III PFC neurons and a reduced number of branching points (FIG. 15C) in all gp120-treated rats; no changes in the average dendrite length were found (FIG. 15D). These data suggest that the structural changes induced by the envelope protein in the PFC compromise performance in PFC-dependent tasks.

### Glia Cells are Required for Modulation of Neuronal FHC by HIV-Gp120.

The HIV envelope protein gp120 causes neuronal injury through a variety of mechanisms, including alteration of CXCR4 signaling and dendritic pruning. To test whether changes in neuronal FHC may be responsible for gp120-mediated neuronal dysfunction, rat cortical neurons in culture were exposed to either the X4-using gp120_{IIIB} or the R5-using gp120_{BaL} (200 pM for 3, 6, or 24 hours). Neuronal levels of FHC protein were assessed by Western blot analysis. For these studies, morphine was used as positive control, due to its ability to increase FHC in pure neuronal cultures as well as neuronal/glial co-cultures. Unlike morphine, gp120_{IIIB} only transiently increased FHC in pure neuronal cultures (FIG. 11A), while the R5-using gp120_{BaL} completely failed to affect neuronal FHC protein levels (FIG. 11B) under the same experimental conditions. However, either type of gp120 (200 pM, up to 24 hr) was able to increase neuronal expression of FHC when neurons were exposed to these proteins in the presence of their glial feeder layer (FIG. 2C; only data for IIIB reported), suggesting that a secreted, and likely unstable, mediator is driving this effect.

### TNF-α and IL-1β Increase Protein Levels of Neuronal FHC.

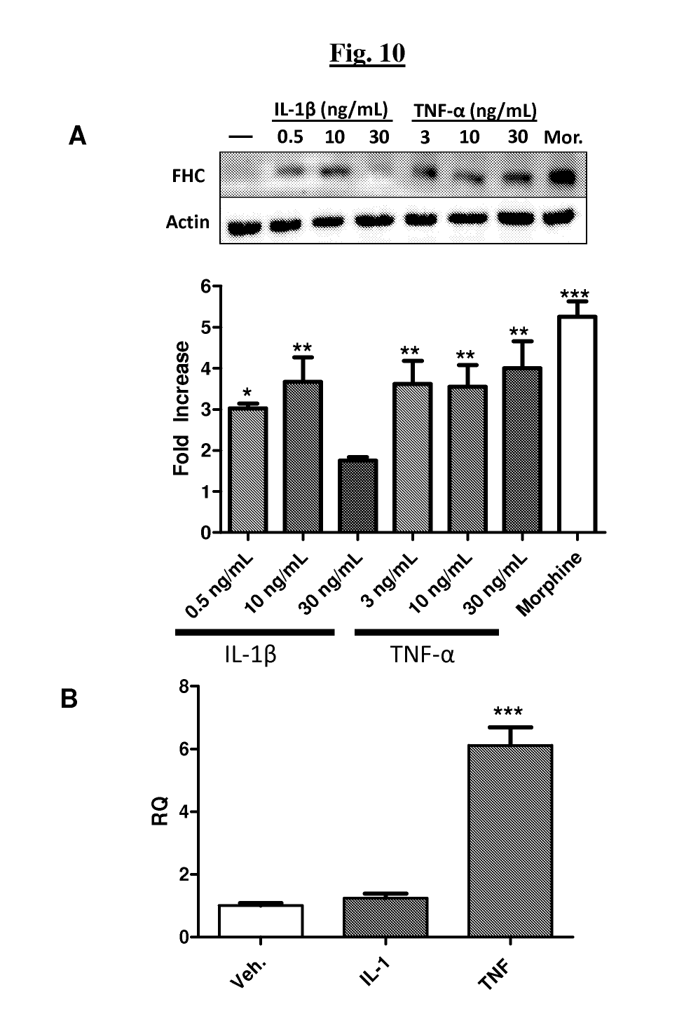

The possibility that a pro-inflammatory cytokine, such as TNF-α and/or IL-1β, directly regulates FHC expression in neurons was investigated. Rat cortical neurons were exposed to either TNF-α or IL-1β in the absence of glia. A concentration range of 0.5 to 30 ng/mL was selected. As shown in FIG. 10, treatment with either cytokine induced a significant increase of FHC protein levels compared to vehicle-treated controls. Maximal responses were not significantly different than those evoked by morphine (FIG. 10A) used as a positive control. Furthermore, differences were noted between the two cytokines: FHC transcript levels increased following TNF-α, but not IL-1β, treatment (FIG. 10B). These experiments suggest that these two cytokines may play an important role in the induction of FHC observed during HIV infection, albeit via different mechanisms.

### X4- and R5-Tropic Gp120 Induced Secretion of IL-1β and TNF-α in Neuronal/Glial Co-Cultures.

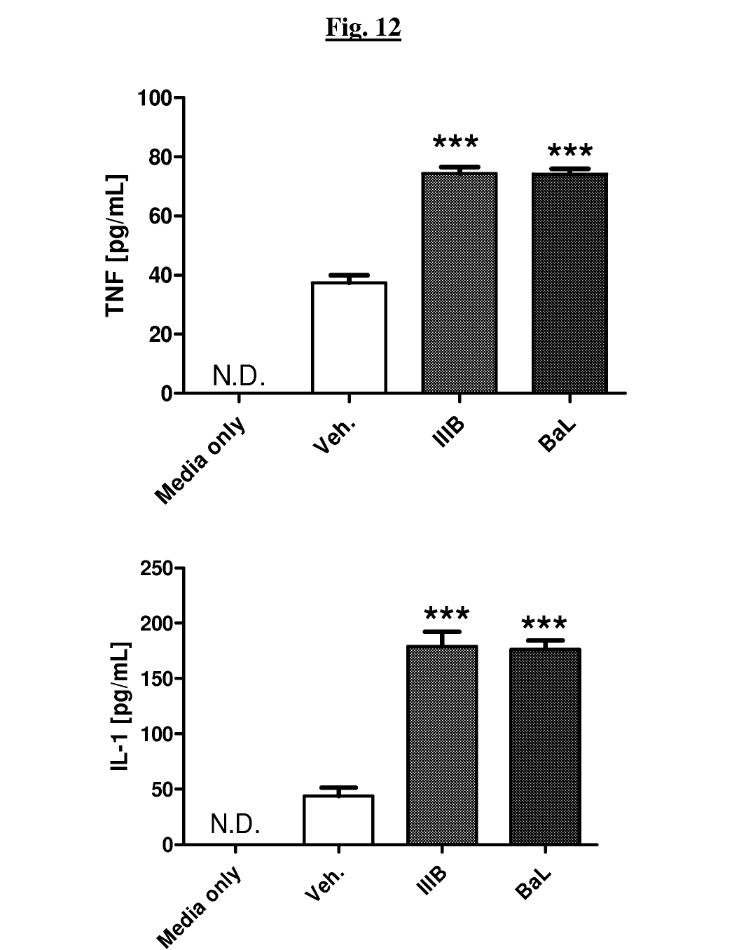

IL-1β and TNF-α are generally increased during HIV-1 infection and chronic inflammation persists in HIV patients despite cART. Glia cells are a main source of cytokines in the CNS. In view of the present finding that these cells are required for gp120's effect on neuronal FHC, IL-1β and TNF-α may be involved in gp 120-induced upregulation of FHC. Therefore, levels of the two cytokines were measured in the co-cultures following exposure to either gp120_{IIIB} or gp120_{BaL} (200 pM for 24 hours). According to these ELISA studies, levels of IL-1β and TNF-α were significantly higher in the culture media of gp 120-treated cultures as compared to controls; the magnitude of the response induced by the X4 and R5 envelope protein was similar (FIG. 12).

### IL-1β is Responsible for Gp120-Induced Upregulation of Neuronal FHC.

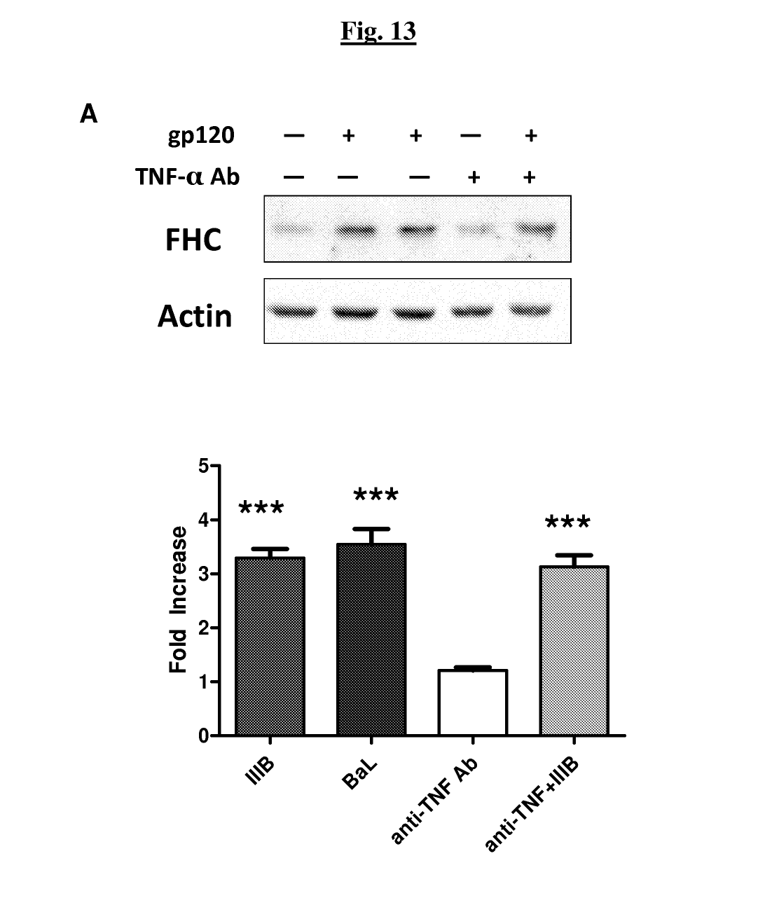

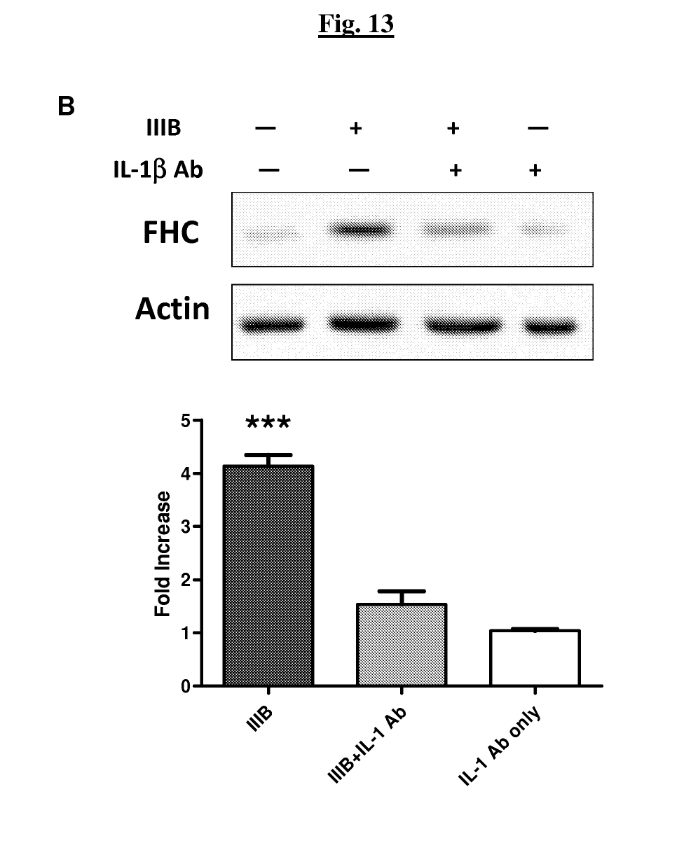

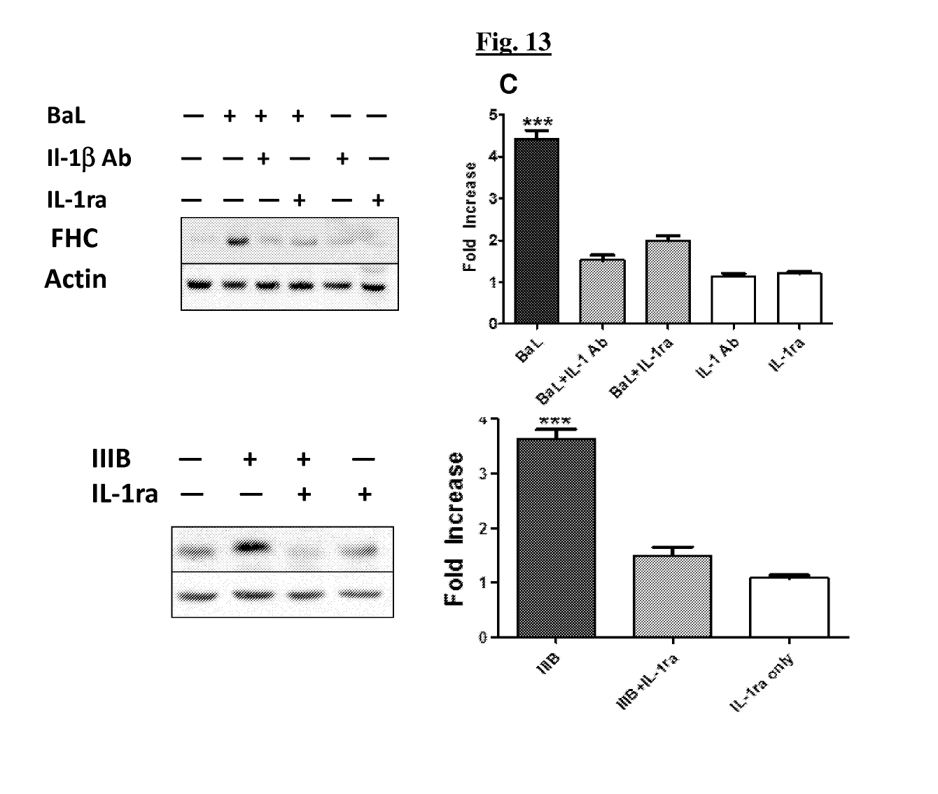

Both IL-1β and TNF-α are candidates for the secreted mediator responsible for gp120's ability to increase neuronal FHC. However, despite TNF-α being secreted in the co-cultures, a neutralizing TNF-α antibody had no effect on upregulation of neuronal FHC by gp120_{IIIB} (FIG. 13A) or gp120_{BaL}. The role of IL-1β in gp120-mediated increases in FHC was then tested. The presence of an IL-1β neutralizing antibody in gp120_{IIIB} (or gp120_{BaL}) exposed co-cultures significantly reduced levels of neuronal FHC to nearly control values (FIGS. 13B-13C). In order to verify the involvement of IL-1β receptor, the IL-1 receptor antagonist, IL-1ra, an endogenous inhibitor of IL-1 signaling, was also utilized. Similar to the neutralizing antibody, IL-1ra completely blocked gp120's ability to upregulate protein levels of neuronal FHC (FIG. 13C). Taken together, these findings highlight the importance of IL-1β in mediating the upregulation of neuronal FHC protein following exposure to gp120, independently of co-receptor (CXCR4/CCR5) preference.

TABLE 1
Clinical correlations within the human cohort
Clinical measures of disease status, including cerebrospinal fluid (CSF)
viral load, peripheral blood (PB) viral load, CD4 cell count, CD8 cell
count, and Memorial Sloan Kettering (MSK) score were compared with
neuronal FHC OD. MSK scores range from 0 to 4, where 0 = no cognitive
impairment, 0.5 = sub-clinical impairment, 1 = mild impairment, 2 =
moderate impairment, 3 = severe impairment, and 4 = profound impairment.
The only significant correlation found between any of these measurements
in our preliminary analysis was between FHC OD and MSK score, sug-
gesting a relationship between FHC levels and cognitive impairment.

| | | Neuronal | CSF viral | PB viral | CD4 cell | CD8 cell | MSK |
|---|---|---|---|---|---|---|---|
| | | FHC | load | load | count | count | score |
| Neuronal | Correlation | — | — | — | — | — | — |
| FHC | Significance | | | | | | |
| | N | | | | | | |
| CSF viral | Correlation | −0.270 | — | — | — | — | — |
| load | Significance | 0.730 | | | | | |
| | N | 4 | | | | | |
| PB viral | Correlation | 0.240 | 0.380 | — | — | — | — |
| load | Significance | 0.646 | 0.162 | | | | |
| | N | 6 | 15 | | | | |
| CD4 cell | Correlation | −0.806 | −0.153 | 0.046 | — | — | — |
| count | Significance | 0.053 | 0.586 | 0.954 | | | |
| | N | 6 | 15 | 4 | | | |
| CD8 cell | Correlation | Insufficient | −0.466 | 0.406 | 0.046 | — | — |
| count | Significance | data | 0.691 | 0.594 | 0.956 | | |
| | N | | 3 | 4 | 4 | | |
| MSK | Correlation | 0.420 | 0.285 | −0.153 | −0.201 | 0.429 | — |
| score | Significance | 0.023 | 0.303 | 0.544 | 0.423 | 0.571 | |
| | N | 29 | 15 | 18 | 18 | 4 | |

TABLE 2
Human tissue cohort
Postmortem human tissue was collected from three sources: the
National NeuroAIDS Tissue Consortium, National Development
and Research Institutes (NDRI), and the Drexel University
Department of Pathology. Patient samples were grouped based
on illicit drug use (all including opiate use) and HIV status.
Neurocognitive impairment was also quantified for each patient,
using the Memorial Sloan Kettering (MSK) scale. This scale
ranges from 0 to 4, where 0 = no cognitive impairment,
0.5 = sub-clinical impairment, 1 = mild impairment,
2 = moderate impairment, 3 = severe impairment, and
4 = profound impairment.

| | | | HAND | |
|---|---|---|---|---|
| Total Patients | Drug Use | HIV Status | (MSK Score) | |
| N = 51 | DU− | HIV− | | |
| | N = 33 | N = 14 | | |
| | | HIV+ | 0-0.5 | N = 6 |
| | | N = 19 | 1-2 | N = 12 |
| | | | 3-4 | N = 1 |
| | DU+ | HIV− | | |
| | N = 18 | N = 7 | | |
| | | HIV+ | 0-0.5 | N = 6 |
| | | N = 11 | 1-2 | N = 5 |
| | | | 3-4 | N = 0 |

TABLE 3
Human subject demographics, disease group, and clinical data at time of death
Patients 1 to 21 are HIV negative; patients 1 to 14 and 22 to 40 did
not abuse drugs; all DU but 2 (marked by an asterisk in table) abused
opiates; other substances of abuse were: cocaine (13/18), alcohol (13/18),
cannabis (9/18), stimulants (7/18); sedatives and hallucinogens (6/18).

| | | | | | | CD4 count | CSF VL | Plasma VL |
|---|---|---|---|---|---|---|---|---|
| | Group | MSK | Race | Sex | Age | (cells/mm³) | (copies/mL) | (copies/mL) |
| 1 | Control | Normal | W/Cau | M | 66 | | — | — |
| 2 | Control | Normal | W/Cau | F | 73 | | — | — |
| 3 | Control | Normal | W/Cau | F | 61 | | — | — |
| 4 | Control | Normal | W/Cau | M | 44 | | — | — |
| 5 | Control | Normal | W/Cau | M | 49 | | — | — |
| 6 | Control | Normal | W/Cau | M | 65 | | — | — |
| 7 | Control | Normal | W/Cau | M | 63 | | — | — |
| 8 | Control | Normal | W/Cau | M | 43 | | — | — |
| 9 | Control | Normal | W/Cau | M | 52 | | — | — |
| 10 | Control | Normal | Black/AA | F | 30 | | — | — |
| 11 | Control | Normal | Unknown | M | 64 | | — | — |
| 12 | Control | Normal | Black/AA | F | 61 | | — | — |
| 13 | Control | Normal | W/Cau | F | 54 | | — | — |
| 14 | Control | Normal | Black/AA | M | 56 | | — | — |
| 15 | DU+ | Normal | W/Cau | M | 54 | | — | — |
| 16 | DU+ | Normal | W/Cau | M | 61 | | — | — |
| 17 | DU+ | Normal | Black/AA | M | 57 | | — | — |
| 18 | DU+ | Normal | Hispanic | M | 47 | | — | — |
| 19 | DU+ | Normal | Hispanic | M | 47 | | — | — |
| 20 | DU+ | Normal | Hispanic | F | 47 | | — | — |
| 21 | DU+ | Normal | Unknown | F | 68 | | — | — |
| 22 | HIV+ | Normal | W/Cau | M | 44 | 147 | | 50 |
| 23 | HIV+ | Normal | W/Cau | M | 34 | | | |
| 24 | HIV+ | Normal | W/Cau | M | 38 | | | |
| 25 | HIV+ | Sub-clin. | W/Cau | M | 64 | 61 | 7,484 | 75 |
| 26 | HIV+ | Sub-clin. | Black/AA | M | 44 | 234 | 618 | 16,909 |
| 27 | HIV+ | Sub-clin. | W/Cauc | M | 59 | | | |
| 28 | HIV+ | Mild | W/Cau | M | 50 | 3 | <50 | 750,000 |
| 29 | HIV+ | Mild | W/Cau | M | 36 | 27 | 670 | 18,985 |
| 30 | HIV+ | Mild | Black/AA | F | 48 | 36 | 1,543 | 380,189 |
| 31 | HIV+ | Mild | Other | M | 45 | 26 | 161 | 316,227 |
| 32 | HIV+ | Mild | W/Cau | M | 38 | | | |
| 33 | HIV+ | Moderate | W/Cau | F | 34 | 14 | 76 | 15,906 |
| 34 | HIV+ | Moderate | W/Cau | M | 42 | 2 | 1,222,799 | 287,582 |
| 35 | HIV+ | Moderate | Other | F | 35 | 360 | 96 | 436,515 |
| 36 | HIV+ | Moderate | Black/AA | F | 34 | | | |
| 37 | HIV+ | Moderate | W/Cau | M | 34 | | | |
| 38 | HIV+ | Moderate | Black/AA | F | 34 | | | |
| 39 | HIV+ | Moderate | Black/AA | M | 37 | | | |
| 40 | HIV+ | Profound | W/Cau | M | 38 | 43 | 1,237,903 | 1,843 |
| 41 | DU+HIV+ | Normal | W/Cau | F | 30 | 407 | | 187 |
| 42 | DU+HIV+ | Normal | Hispanic | F | 46 | 88 | 1,305 | 74,294 |
| 43 | DU+HIV+ | Normal | W/Cau | M | 46 | 3 | 201 | 779,000 |
| 44 | DU+HIV+ | Normal | W/Cau | M | 37 | | | |
| 45 | DU+HIV+ | Sub-clin. | Other | F | 44 | 66 | 249 | 750,000 |
| 46 | DU+HIV+ | Sub-clin. | W/Cau | M | 56 | | | |
| 47 | DU+HIV+ | Mild | W/Cau | M | 54 | 211 | | 400 |
| 48 | DU+HIV+ | Mild | Other | F | 51 | | | |
| 49 | DU+HIV+ | Mild | W/Cau | M | 32 | 49 | 3,140,694 | 972,503 |
| 50 | DU+HIV+ | Mild | W/Cau | M | 39 | | | |
| 51 | DU+HIV+ | Moderate | W/Cau | M | 43 | 44 | <50 | 65 |

TABLE 4
Non-human primate tissue cohort
Prefrontal cortex was obtained from rhesus macaques that under-
went 4 types of treatment: Control (n = 11), Morphine (n = 3),
SIV (n = 4), and Morphine + SIV (n = 2). Animals were randomly
assigned to treatment groups, without regard to sex, age, or weight.

| | | | | | Mor- | Weight at |
|---|---|---|---|---|---|---|
| | ID | Sex | Age | SIV | phine | necropsy (kg) |
| Control | CL14 | F | 10 | − | − | 5.2 |
| | DP27 | F | 9 | − | − | 7.2 |
| | FE82 | F | 7 | − | − | 6.6 |
| | FM11 | M | 8 | − | − | 10.2 |
| | FV39 | M | 9 | − | − | 10.9 |
| | GA15 | M | 6 | − | − | 12.2 |
| | HM63 | M | 3 | − | − | 3.6 |
| | HN64 | M | 3 | − | − | 4.9 |
| | HP24 | M | 3 | − | − | 3.5 |
| | IP37 | F | 16 | − | − | 8.7 |
| | IP43 | F | 6 | − | − | 5.4 |
| Morphine | 4684 | F | 5 | − | + | 6.3 |
| | 4690 | M | 3 | − | + | 5.1 |
| | 4697 | M | 3 | − | + | 4.4 |
| SIV | 4678 | F | 4 | + | − | 3.6 |
| | 4679 | F | 3 | + | − | 5.6 |
| | 4680 | F | 5 | + | − | 4.9 |
| | 4688 | M | 3 | + | − | 4.9 |
| Morphine/SIV | 4692 | F | 3 | + | + | 4.2 |
| | 4693 | F | 3 | + | + | 2.9 |

TABLE 5
Physiological properties of layer ⅔ pyramidal neurons exposed to CXCL12, AMD3100 or their respective vehicles
There were no significant differences between drug and vehicle conditions (all p > 0.05).

| | resting | | | | | spike | |
|---|---|---|---|---|---|---|---|
| | membrane | input | spike | peak spike | after | half- | 20-80% |
| | potential | resistance | threshold | amplitude | hyperpolarization | width | rise time |
| | (mV) | (M′Ω) | (mV) | (mV) | (mV) | (ms) | (ms) |
| vehicle_{AMD} | −79.6 ± 1.0 | 149.3 ± 13.3 | −41.8 ± 1.5 | 75.2 ± 3.3 | −12.1 ± 1.3 | 1.2 ± 0.06 | 0.28 ± 0.02 |
| AMD3100 | −76.9 ± 2.3 | 138.3 ± 9.5 | −38.4 ± 3.0 | 72.6 ± 5.3 | −12.8 ± 1.5 | 1.3 ± 0.12 | 0.31 ± 0.02 |
| vehicle_{CXCL12} | −75.82 ± 1.9 | 124.2 ± 13.6 | −40.4 ± 1.1 | 72.4 ± 1.8 | −12.7 ± 1.0 | 1.2 ± 0.9 | 0.28 ± 0.01 |
| CXCL12 | −76.1 ± 2.3 | 121.8 ± 13.2 | −42.9 ± 2.3 | 74.0 ± 2.8 | −12.0 ± 0.9 | 1.2 ± 0.07 | 0.29 ± 0.01 |

TABLE 6
Animals tested in flexible
attention task following
gp120 treatment

| | gp120 | BSA |
|---|---|---|
| SD (n = 11) | 5 | 6 |
| F344 (n = 6) | 5 | 1 |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating or preventing HAND in a subject in need thereof, wherein the method comprises administering to the subject a therapeutically effective amount of an agent that down-regulates FHC in the subject, whereby HAND is treated or prevented in the subject.

2. The method of claim 1, wherein the agent is selected from the group consisting of an antibody, siRNA, ribozyme, antisense, aptamer, peptidomimetic, iron chelating agent, and any combinations thereof.

3. The method of claim 2, wherein the antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

4. The method of claim 1, wherein the subject is a mammal.

5. The method of claim 4, wherein the mammal is human.

6. The method of claim 1, wherein the agent is administered by an inhalational, oral, rectal, vaginal, parenteral, topical, transdermal, pulmonary, intranasal, buccal, ophthalmic, intrathecal, intracranial, or intravenous route of administration.

7. The method of claim 1, wherein the subject is further administered at least one anti-HIV drug.

8. The method of claim 7, wherein the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

9. A method of treating or preventing HAND in a subject in need thereof, wherein the method comprises administering to the subject a therapeutically effective amount of an agent that decreases that concentration, expression level and/or activity of IL-1β in the subject, whereby HAND is treated or prevented in the subject.

10. The method of claim 9, wherein the agent is selected from the group consisting of an antibody, siRNA, ribozyme, antisense, aptamer, peptidomimetic, iron chelating agent, and any combinations thereof.

11. The method of claim 9, wherein the agent is at least one selected from the group consisting of interleukin-1 receptor antagonist (IL-1ra), anakinra and rilonacept.

12. The method of claim 10, wherein the antibody comprises an antibody selected from the group consisting of a polyclonal antibody, monoclonal antibody, humanized antibody, synthetic antibody, heavy chain antibody, human antibody, biologically active fragment of an antibody, and any combinations thereof.

13. The method of claim 9, wherein the subject is a mammal.

14. The method of claim 13, wherein the mammal is human.

15. The method of claim 9, wherein the agent is administered by an inhalational, oral, rectal, vaginal, parenteral, topical, transdermal, pulmonary, intranasal, buccal, ophthalmic, intrathecal, intracranial or intravenous route of administration.

16. The method of claim 9, wherein the subject is further administered at least one anti-HIV drug.

17. The method of claim 16, wherein the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

18. A method of treating or preventing in a subject in need thereof a disease or condition associated with CXCR4 up-regulation, wherein the method comprises administering to the subject a therapeutically effective amount of an opioid agonist, whereby the disease or condition is treated or prevented.

19. The method of claim 18, wherein the disease or condition comprises cancer, metastasis, liver fibrosis, HIV infection or pain.

20. The method of claim 19, wherein liver fibrosis includes HIV-associated liver fibrosis.

21. The method of claim 18, wherein the opioid agonist is at least one selected from the group consisting of adrenorphin, amidorphin, casomorphin, DADLE, DAMGO, dermorphin, endomorphin, morphiceptin, octreotide, opiorphin, TRIMU 5, codeine, morphine, thebaine, oripavine, esters of morphine, ethers of morphine, semi-synthetic alkaloid derivatives, anilidopiperidines, phenylpiperidines, diphenylpropylamine derivatives, benzomorphan derivatives, oripavine derivatives, morphinan derivatives, lefetamine, menthol, meptazinol, mitragynine, tilidine, tramadol and tapentadol.

22. The method of claim 18, wherein the subject is a mammal.

23. The method of claim 22, wherein the mammal is human.

24. The method of claim 18, wherein the opioid antagonist is administered by an inhalational, oral, rectal, vaginal, parenteral, topical, transdermal, pulmonary, intranasal, buccal, ophthalmic, intrathecal, intracranial or intravenous route of administration.

25. The method of claim 18, wherein the disease or condition comprises HIV infection and the subject is further administered at least one anti-HIV drug.

26. The method of claim 25, wherein the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.


SEQUENCE LISTING (1 sequence)

SEQ ID NO: 1 - protein, 5 aa, Artificial Sequence
  Chemicaly Synthesized; DADLE or [DAla2-DLeu5]- enkephalin
YXGFX